SEQUENCE LISTING (38 sequences)

SEQ ID NO: 1 - protein, 126 aa, Gallus sp.
AVTLDESGGGLQTPGGGLSLVCKASGFDFSSYAMVWVRQAPGKGLEYVGEITNYAAGSGTWYGAAVKGRATISRDNGQSTVRLQLNNLRAEDTATYYCAKGPISGSIGYLSSIDAWGHGTEVIVSS

SEQ ID NO: 2 - protein, 124 aa, Gallus sp.
AVTLDESGGGLQTPGGALSLVCKASGFDFSSSYINWVRQAPGKGLEYVAQINKDGGGSWYAPVVKGRATISRDNGQSTVRLQLSNLRAEDTGIYFCAKNADSGCVGVGGCIDTWGHGTEVIVSS

SEQ ID NO: 3 - protein, 125 aa, Gallus sp.
AVTLDESGGGLQTPGGALSLVCKASGFTFNSNGMGWVRQAPGKGLEWVAGIHSSGRYTYYGTAVKGRATISRDNGQSTVRLQLNDLRAEDTATYYCAKNADSAYGYWYAGSIDAWGHGTEVIVSS

SEQ ID NO: 4 - protein, 123 aa, Gallus sp.
AVTLDESGGGLQTPGGALSLVCKASGFSFDRHGMFWVRQAPGKGLEWVAGIRSTGSYPKYGPAVKGRATISRDNGQSTVRLQLNNLRAEDTARYFCVRNVYIGDYDGDSIDAWGHGTEVIVSS

SEQ ID NO: 5 - protein, 104 aa, Gallus sp.
ALTQPSSVSANPGETVKITCSGGSSRYGYGWYQQKSPGSAPVTVIYWNDKRPSDIPSRFSGSKSGSTGTLTITGVRAEDEAVYYCGNYDNNDPAFGAGTTLTVL

SEQ ID NO: 6 - protein, 109 aa, Gallus sp.
ALTQPSSVSANPGETVKITCSGGSYYYGGNYYYGWYQQKSPGSAPVTLIYNNNKRPSDIPSRFSGSTSGSTGTLTITGVQADDEAVYYCGGYDSSYVAIFGAGTTLTVL

SEQ ID NO: 7 - protein, 106 aa, Gallus sp.
ALTQPSSVSANLGGTVEITCSGGYSSYGYSWHQQKSPASAPVTLIYNNNNRPSDIPSRFSGSASGSTATLTITGVQAEDEAVYYCAFTDYSSLAGVFGAGTTLTVL

SEQ ID NO: 8 - protein, 108 aa, Gallus sp.
ALTQPSSVSANLGGTVEITCSGSSSGYGSYYGWYQQKSPGSAPVTVIYENTNRPSNIPSRFSGSESGSTATLTITGVQAEDEAVYFCGSADDSGNSAIFGAGTTLTVL

SEQ ID NO: 9 - protein, 6 aa, Gallus sp.
SSYAMV

SEQ ID NO: 10 - protein, 6 aa, Gallus sp.
SSSYIN

SEQ ID NO: 11 - protein, 6 aa, Gallus sp.
NSNGMG

SEQ ID NO: 12 - protein, 6 aa, Gallus sp.
DRHGMF

SEQ ID NO: 13 - protein, 18 aa, Gallus sp.
EITNYAAGSGTWYGAAVK

SEQ ID NO: 14 - protein, 16 aa, Gallus sp.
QINKDGGGSWYAPVVK

SEQ ID NO: 15 - protein, 16 aa, Gallus sp.
GIHSSGRYTYYGTAVK

SEQ ID NO: 16 - protein, 16 aa, Gallus sp.
GIRSTGSYPKYGPAVK

SEQ ID NO: 17 - protein, 16 aa, Gallus sp.
KGPISGSIGYLSSIDA

SEQ ID NO: 18 - protein, 16 aa, Gallus sp.
KNADSGCVGVGGCIDT

SEQ ID NO: 19 - protein, 17 aa, Gallus sp.
KNADSAYGYWYAGSIDA

SEQ ID NO: 20 - protein, 15 aa, Gallus sp.
RNVYIGDYDGDSIDA

SEQ ID NO: 21 - protein, 10 aa, Gallus sp.
SGGSSRYGYG

SEQ ID NO: 22 - protein, 14 aa, Gallus sp.
SGGSYYYGGNYYYG

SEQ ID NO: 23 - protein, 10 aa, Gallus sp.
SGGYSSYGYS

SEQ ID NO: 24 - protein, 12 aa, Gallus sp.
SGSSSGYGSYYG

SEQ ID NO: 25 - protein, 7 aa, Gallus sp.
WNDKRPS

SEQ ID NO: 26 - protein, 7 aa, Gallus sp.
NNNKRPS

SEQ ID NO: 27 - protein, 7 aa, Gallus sp.
NNNNRPS

SEQ ID NO: 28 - protein, 7 aa, Gallus sp.
ENTNRPS

SEQ ID NO: 29 - protein, 9 aa, Gallus sp.
GNYDNNDPA

SEQ ID NO: 30 - protein, 10 aa, Gallus sp.
GGYDSSYVAI

SEQ ID NO: 31 - protein, 11 aa, Gallus sp.
AFTDYSSLAGV

SEQ ID NO: 32 - protein, 11 aa, Gallus sp.
GSADDSGNSAI

SEQ ID NO: 33 - protein, 124 aa, synthetic construct
EVQLVESGGGLVQPGGSLRLSCKASGFTFSSSYINWVRQAPGKGLEYVAQINKDGGGSWYAPVVKGRFTISRDNAKNTVYLQMNSLRAEDTAVYYCAKNADSGCVGVGGCIDTWGQGTTVTVSS

SEQ ID NO: 34 - protein, 110 aa, synthetic construct
SYELTQPSSVSVSPGQTVRITCSGGSYYYGGNYYYGWYQQKPGQAPVTVIYNNNKRPSGIPERFSGSNSGNTGTLTISGVQAEDEADYYCGGYDSSYVAIFGGGTKLTVL

SEQ ID NO: 35 - protein, 297 aa, Homo sapiens
MTTPRNSVNGTFPAEPMKGPIAMQSGPKPLFRRMSSLVGPTQSFFMRESKTLGAVQIMNGLFHIALGGLLMIPAGIYAPICVTVWYPLWGGIMYIISGSLLAATEKNSRKCLVKGKMIMNSLSLFAAISGMILSIMDILNIKISHFLKMESLNFIRAHTPYINIYNCEPANPSEKNSPSTQYCYSIQSLFLGILSVMLIFAFFQELVIAGIVENEWKRTCSRPKSNIVLLSAEEKKEQTIEIKEEVVGLTETSSQPKNEEDIEIIPIQEEEEEETETNFPEPPQDQESSPIENDSSP

SEQ ID NO: 36 - protein, 291 aa, Mus musculus
MSGPFPAEPTKGPLAMQPAPKVNLKRTSSLVGPTQSFFMRESKALGAVQIMNGLFHITLGGLLMIPTGVFAPICLSVWYPLWGGIMYIISGSLLAAAAEKTSRKSLVKAKVIMSSLSLFAAISGIILSIMDILNMTLSHFLKMRRLELIQTSKPYVDIYDCEPSNSSEKNSPSTQYCNSIQSVFLGILSAMLISAFFQKLVTAGIVENEWKRMCTRSKSNVVLLSAGEKNEQTIKMKEEIIELSGVSSQPKNEEEIEIIPVQEEEEEEAEINFPAPPQEQESLPVENEIAP

SEQ ID NO: 37 - DNA, 49 bp, synthetic construct
  source 1..49: mol_type = other DNA
  misc_feature 1..49: note = chVH-Adapt-F primer
acactctttccctacacgacgctcttccgatctgacgcggtgacgttgg

SEQ ID NO: 38 - DNA, 52 bp, synthetic construct
  source 1..52: mol_type = other DNA
  misc_feature 1..52: note = chVH-Adapt-R primer
gactggagttcagacgtgtgctcttccgatctaaacgctcggaccttttgtc

(19) United States
(12) Patent Application Publication
(10) Pub. No.: US 2026/0055202 A1
(43) Pub. Date: Feb. 26, 2026

(54) CHICKEN-DERIVED CD20 ANTIBODIES WITH POTENT B CELL DEPLETION ACTIVITY

(71) Applicant: THE TEXAS A&M UNIVERSITY SYSTEM, College Station, TX (US)

(72) Inventors: Karrupiah CHOCKALINGAM, College Station, TX (US); Zhilei CHEN, College Station, TX (US)

(21) Appl. No.: 19/103,693

(22) PCT Filed: Aug. 17, 2023
(86) PCT No.: PCT/US2023/072360

§ 371 Date: Feb. 13, 2025

Related U.S. Application Data
(60) Provisional application No. 63/398,676, filed on Aug. 17, 2022.

(51) Int. Cl. C07K 16/28

(52) U.S. Cl. CPC C07K 16/2887; C07K 2317/24; C07K 2317/33; C07K 2317/732; C07K 2317/734

(57) ABSTRACT

The present disclosure provides multiple anti-hCD20 mAbs as well as humanization of antibodies. The antibodies described herein are chicken-derived and exhibit significantly enhanced B-cell-specific antibody-dependent cellular cytotoxicity (ADCC) and complement-dependent cytotoxicity (CDC) potency as well as improved depletion of B lymphoma cells in vivo relative to Rituximab.

### CROSS-REFERENCE TO RELATED APPLICATIONS

This application claims the benefit under 35 USC § 119(e) of U.S. Provisional Application Ser. No. 63/398,676, filed on Aug. 17, 2022, the entire disclosure of which is incorporated herein by reference.

### INCORPORATION BY REFERENCE OF MATERIAL SUBMITTED ELECTRONICALLY

Incorporated by reference in its entirety is a computer-readable nucleotide/amino acid sequence listing submitted concurrently herewith and identified as follows: 35.3 kilobytes ACII (xml) file named “78090-393876_SL.xml,” created on Aug. 16, 2023.

### BACKGROUND AND SUMMARY OF THE INVENTION

CD20 is a conserved marker found on B cells. Abnormal B cells are present in many different disease states, including cancers of the blood and autoimmune diseases. CD20 is known to be a validated target for several of these diseases. For instance, therapeutic effects can be achieved by specific depletion/destruction of CD20-positive B cells.

Monoclonal antibodies (mAbs), which typically possess both a targeting function and an ability to trigger biological mechanisms for the destruction of their binding targets, can be particularly useful as therapeutic agents for B cell depletion. For instance, several antibodies (Abs) against human CD20 (hCD20) have been approved for the treatment of lymphoid malignancies and autoimmune diseases, including the first approved anti-cancer mAb therapeutic, Rituximab.

Current anti-hCD20 Abs in the clinic setting are derived from immunization of a mouse host. However, the high (67%) sequence homology between the CD20 ectodomains from mice and humans makes it challenging to discover anti-hCD20 mAbs with new specificities, and ostensibly new therapeutic potential. As a result, the majority of existing anti-hCD20 Abs found via mouse immunization bind a highly similar overlapping epitope on hCD20 encompassing the ANPS motif at position 170-173 within the larger extracellular loop.

However, despite several years of trying to develop improved anti-hCD20 mAb therapeutics, current antibodies Abs remain only partially effective in patients. Current therapies often require extended treatment and/or combination with chemotherapy or other immunotherapies/targeted agents. Moreover, most patients develop recurrent disease that is refractory to retreatment and as many as 50% of patients fail to show any response. Therefore, there exists an urgent need to develop new anti-hCD20 Abs with an enhanced ability to deplete B cells, particularly malignant cells, as therapies for patients.

Accordingly, the present disclosure provides new compositions and methods utilizing novel antibodies that target CD20. As described herein, novel antibodies were identified that can bind hCD20 epitopes not limited to the 33% of sequence space divergent between humans and mice and that exhibit enhanced B cell depletion activity, by utilizing a phylogenetically distant species (chickens) as a host for immunization.

The present disclosure provides multiple anti-hCD20 mAbs as well as humanization of antibodies. The antibodies described herein are chicken-derived and exhibit significantly enhanced B-cell-specific antibody-dependent cellular cytotoxicity (ADCC) and complement-dependent cytotoxicity (CDC) potency as well as improved depletion of B lymphoma cells in vivo relative to Rituximab.

Other objects, features and advantages of the present disclosure will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### BRIEF DESCRIPTION OF THE DRAWINGS

FIG. 1 shows protein sequences of the variable regions of the chicken-derived Abs AC1, AC11, ACR7, and ACR8. Complementarity-determining region (CDR) sequences for each sequence are bolded and underlined.

FIG. 2 shows protein sequences of the variable regions of the humanized mAb hAC11-10 in alignment with that of the parental (chicken) mAb AC11. Complementarity-determining region (CDR) sequences for each sequence are bolded and underlined.

FIG. 3 shows representative flow cytometry histograms showing binding of anti-hCD20 Abs to Raji cells. Median fluorescence intensities (MFIs) from histograms calculated using FlowJo software were used to generate the binding curves in FIG. 8. The identity and concentration of the Ab and calculated MFI is shown in the insets of histograms.

FIG. 4 shows flow cytometric evaluation of binding of Fab-displaying M13 bacteriophage to enhanced green fluorescent protein (EGFP)-hCD20-(upper) or EGFP-transfected (lower) CHO cells showing that phage displaying the Fab portions of the four chicken-derived Abs bind specifically to hCD20 in a manner dependent on the expression of the EGFP-hCD20 fusion protein. Phage binding was detected by Alexa Fluor 647 (AF647)-labeled anti-M13 Ab. Data are from one of two independent, representative experiments.

FIG. 5A shows flow cytometric analysis gating strategy for whole-blood B cell depletion assay. Representative side-scatter vs. forward scatter dot plot of human whole blood following mAb treatment and staining with labeled B-cell- and T-cell-specific Abs. 10,000 lymphocyte events (within the black gate) were collected and analysed per sample. All flow cytometric analyses were performed directly on mAb-treated/stained samples subjected to red blood cell lysis using 1×BD FACS Lysing Solution as described in the main text. Q1: B cells; Q3: T cells.

FIG. 5B shows flow cytometric analysis gating strategy for whole-blood B cell depletion assay. Representative P E vs. A647 fluorescence dot plot of mAb-treated whole blood incubated with PE-labeled anti-CD19 and Atto 647 (A647)-labeled anti-CD3 Abs. All flow cytometric analyses were performed directly on mAb-treated/stained samples subjected to red blood cell lysis using 1×BD FACS Lysing Solution as described in the main text. Q1: B cells; Q3: T cells.

FIG. 5C shows flow cytometric analysis gating strategy for whole-blood B cell depletion assay. Representative P E vs. A647 fluorescence dot plot of mAb-treated whole blood incubated with PE- and A647-labeled isotype-control Abs. All flow cytometric analyses were performed directly on mAb-treated/stained samples subjected to red blood cell lysis using 1×BD FACS Lysing Solution as described in the main text. Q1: B cells; Q3: T cells.

FIG. 6A shows Location of primer annealing sites for full-length Fab amplification and cloning into the BsaI sites of the phagemid pGBid. BidP: bi-directional promoter. HCDR3-nested primer strategy for construction of full-length sequences of rare enriched anti-hCD20 Fab candidates identified by next-generation sequencing. The 4th-round-enriched phagemid library depleted of the dominant Fab clone AC1 by restriction enzyme digestion was used as a template for PCR amplification of overlap extension (OE) PCR fragments.

FIG. 6B shows primer pairs used for amplification of input fragments for OE PCR. HCDR3-nested primer strategy for construction of full-length sequences of rare enriched anti-hCD20 Fab candidates identified by next-generation sequencing. The 4th-round-enriched phagemid library depleted of the dominant Fab clone AC1 by restriction enzyme digestion was used as a template for PCR amplification of overlap extension (OE) PCR fragments.

FIG. 7 shows the primers used in the examples. Illumina adapter sequences are underlined.

FIG. 8 shows antibody binding to B lymphoma cells. Raji cells were incubated with anti-hCD20 Abs followed by staining with 50 μg/mL Atto 488-labeled anti-human Fe Ab and flow cytometric evaluation of binding. The median fluorescence intensity (MFI) of Ab-coated cells was determined using FlowJo software. Data for the lowest mAb concentration derives from cells incubated with just buffer (no Ab). Binding of anti-hCD20 Abs was also evaluated via direct immunofluorescence using Atto 488-labeled anti-hCD20 Abs (FIG. 9A). Values are the mean±SD of duplicate measurements. Data are from one of two independent, representative experiments and were fitted to an asymmetric sigmoidal 5PL curve using GraphPad Prism. Statistical analysis by unpaired t test: AC1, ACR8 vs. RTX is significant (P<0.01) for all the non-zero mAb concentrations (5×10⁻⁹ g/mL in the chart represents zero mAb); statistical significance of AC11, ACR7 over RTX is indicated by asterisks with the same color designation as the Ab. *P<0.05, **P<0.01.

FIG. 9A shows antibody binding to B lymphoma cells as measured via direct immunofluorescence. Raji cells were incubated with A488-labeled anti-hCD20 Abs followed by washing and flow cytometric analysis using a BD LSRFortessa X-20 instrument. The median fluorescence intensities (MFIs) of Ab-coated cells were determined using FlowJo software and fitted to an asymmetric sigmoidal 5PL curve using GraphPad Prism. Data are the mean±SD of duplicate measurements and are from one of two independent, representative experiments. Statistical analysis: the difference between ACR8 and RTX is significant (P<0.01) for all mAb concentrations ≥0.50 μg/mL (log [mAb] of −6.3); the difference between AC1 and RTX is significant (P<0.05) for all mAb concentrations ranging from 0.50 μg/mL (log [mAb] of −6.3) to 10 μg/mL (log [mAb] of −5.0). *P<0.05; **P<0.01, ***P<0.001, by unpaired t test.

FIG. 9B shows the degree of labeling (DOL) of the A488-labeled Abs spectrophotometrically determined using a NanoDrop 1000 instrument per the Thermo Fisher Amine-Reactive Probes manual (mp00143). In order to rule out the possibility of aggregates of labeled Abs giving rise to an enhanced binding signal, an attempt was made to detect aggregates via dynamic light scattering (DLS) measurement using a Malvern Zetasizer Nano ZS instrument.

FIG. 10A shows humanization of AC11. ADCC activation in FcγRIIIa(V158)-expressing pseudoeffector Jurkat cells using Raji target cells was measured using the ADCC Reporter Bioassay (Promega) and the humanized variant hAC11-10. Data are mean±SD of duplicate measurements and are from one of two independent, representative experiments. Statistical significance of hAC11-10 over AC11 was determined by unpaired t test.

FIG. 10B shows humanization of AC11 and whole blood B cell depletion activity of the parental chicken-human chimeric mAb AC11 and the humanized variant hAC11-10. Data are mean±SD of duplicate measurements and are from one of two independent, representative experiments. Statistical significance of hAC11-10 over AC11 was determined by unpaired t test.

FIG. 11 shows Raji cell binding/hCD20 receptor occupancy (RO) of the humanized chicken-derived anti-hCD20 mAb hAC11-10, and the clinically used type II anti-hCD20 mAb Obinutuzumab (humanized, afucosylated) and type I anti-hCD20 mAb Rituximab (mouse-human chimeric). The experiment was carried out by detection of bound unlabeled anti-hCD20 Abs using A488-labeled secondary anti-human IgG Fcγ Ab. Median fluorescence values from flow cytometry were obtained using FlowJo software. EC₅₀ values were generated from an asymmetric sigmoidal 5PL fit using GraphPad Prism. Data are from one of two independent, representative experiments. Statistical analysis comparing hAC11-10 to Obinutuzumab is shown for the two highest mAb concentrations, 16.7 μg/mL and 50 μg/mL (*P=0.015 for both), and comparing hAC11-10 to Rituximab is shown for one concentration, 5.6 μg/mL. All statistical analyses were by unpaired t test.

FIG. 12A shows antibody-dependent cellular cytotoxicity (ADCC) of anti-CD20 Abs. (A) PBMC ADCC bioassay. Raji B lymphoma target cells expressing a HiBiT fusion protein were incubated with anti-CD20 Abs and human PBMCs (V158/F158 mixed genotype) as effectors (E:T, 25:1). HiBiT release following Ab-mediated cell lysis was quantified via luminescence measurement and expressed as a percentage of the lysis signal obtained with 100 μg/mL digitonin treatment. Statistical significance over RTX is indicated by asterisks with the same color designation as the Ab. Black asterisks designate statistical significance over RTX for all Abs. *P<0.05. Values are the mean±SD of duplicate measurements.

FIG. 12B shows antibody-dependent cellular cytotoxicity (ADCC) of anti-CD20 Abs. (B) ADCC reporter bioassay. Raji cells were incubated with anti-CD20 Abs and engineered Jurkat pseudoeffector cells stably expressing the FcγRIIIa receptor, V158 (high affinity) variant, and an NFAT response element driving expression of firefly luciferase. Activation of ADCC signaling in the engineered effector cells was measured via the luciferase reporter. Data are from one of two independent, representative experiments and were fitted to a sigmoidal 4PL curve using GraphPad Prism. Statistical significance over RTX: P<0.05 for the two extreme mAb concentrations (3.6×10⁻¹¹ g/mL, 1.0×10⁻⁵ g/mL), P<0.01 for the remaining concentrations. All analyses were by unpaired t test. RTX, Rituximab; OBZ, Obinutuzumab (afucosylated); RLU: Relative Luminescence Units.

FIG. 13 shows complement-dependent cytotoxicity of anti-CD20 Abs. Raji cells were incubated with anti-CD20 Abs in the presence of 16% fresh human serum at 37° C. for 2 hr and cell viability was colorimetrically quantified using WST-1 cell proliferation reagent. Data for the lowest mAb concentration derives from cells treated with 50 μg/mL isotype control. Values are the mean±SD of at least duplicate measurements. Data are from one of two independent, representative experiments and were fitted to an asymmetric sigmoidal 5PL curve using GraphPad Prism. Statistical significance over RTX is indicated by asterisks with the same color designation as the Ab. Black asterisk designates statistical significance over RTX for all Abs. *P<0.05, **P<0.01. Statistical analysis for OFA comparison: AC1 and AC11, P>0.05 for 1.22×10−7 g/mL and lower concentrations; ACR7, P>0.05 for all concentrations except 1.35×10⁻⁶, 4.50×10⁻⁶, 5.00×10⁻⁵ g/mL; ACR8, P>0.05 for all mAb concentrations except for 4.50×10⁻⁶ g/mL; RTX, P<0.05 for 1.22×10⁻⁷ g/mL and higher concentrations. Analyses by unpaired t test. OFA, Ofatumumab.

FIG. 14A shows whole blood B cell depletion activity of anti-hCD20 mAbs. Anti-hCD20 Abs were incubated with unmodified fresh human whole blood. The Ab/blood mixtures were incubated at 37° C./5% CO₂ for 24 h and B cell depletion was flow cytometrically monitored. Dose response of mAbs AC1 and AC11 using whole blood from four different donors are shown. Statistical significance over RTX is indicated by asterisks with the same color designation as the Ab. All values are the mean±SD of duplicate measurements. *P<0.05; **P<0.01, ***P<0.001, ****P<0.0001, ns, not significant, by unpaired t test.

FIG. 14B shows whole blood B cell depletion activity of anti-hCD20 mAbs. Anti-hCD20 Abs were incubated with unmodified fresh human whole blood. Dose response of mAbs ACR7 and ACR8 utilizing blood from two different donors are shown. Statistical significance over RTX is indicated by asterisks with the same color designation as the Ab. All values are the mean±SD of duplicate measurements. *P<0.05; **P<0.01, ***P<0.001, ****P<0.0001, ns, not significant, by unpaired t test.

FIG. 14C shows whole blood B cell depletion activity of anti-hCD20 mAbs. Anti-hCD20 Abs were incubated with whole blood in which the plasma was collected by centrifugation and incubated at either 56° C. or ambient temperature for 30 mins prior to remixing with the original blood cell pellet. B cell depletion at either 1 μg/mL (Rituximab) or 0.01 μg/mL (all other mAbs) in whole blood containing either heat-treated (HT) plasma or non-heat-treated (NHT) plasma. Data for two different blood donors are shown. Statistical significance over RTX is indicated by asterisks with the same color designation as the Ab. All values are the mean±SD of duplicate measurements. *P<0.05; **P<0.01, ***P<0.001, ****P<0.0001, ns, not significant, by unpaired t test.

FIG. 15A shows direct cell death effects (DCDE) induced by anti-hCD20 Abs. Raji cells (3.7×10⁵ cells in 570 L) seeded in the internal wells of 24-well plates in the presence of 10 μg/mL anti-hCD20 mAbs or Trastuzumab (isotype control). The plates were incubated at 37° C./5% CO₂ for 24 hours prior to analysis. Analysis using a Beckman Coulter Vi-CELL XR Cell Viability Analyzer. Cell health was gauged by either the percentage of cells able to exclude the Trypan Blue dye (left), or total viable cell counts (right). Data are the mean±SD of triplicate measurements and are from one of two independent, representative experiments. *P<0.05; **P<0.01, ***P<0.001, ****P<0.0001 by unpaired t test.

FIG. 15B shows direct cell death effects (DCDE) induced by anti-hCD20 Abs. Raji cells (3.7×10⁵ cells in 570 L) seeded in the internal wells of 24-well plates in the presence of 10 μg/mL anti-hCD20 mAbs or Trastuzumab (isotype control). The plates were incubated at 37° C./5% CO₂ for 24 hours prior to analysis. Analysis by CellTiter-Glo Luminescent Cell Viability Assay (Promega), which measures viable cells based on quantitation of the ATP present. CellTiter-Glo (CTG) reagent was diluted 10-fold in reverse-osmosis water and 50 μL of the diluted reagent was added to 20 μL of the Ab-treated cells in a 96-well plate. The plate was agitated at 800 rpm for 2.5 and incubated at ambient temperature for 10 minutes. 20 μL of the CTG-lysed cell solutions were transferred to an opaque white 96-well plate and luminescence was measured with a Cytation 5 plate reader (BioTek) using a 0.2 s integration time. Data are the mean±SD of triplicate measurements and are from one of two independent, representative experiments. *P<0.05; **P<0.01, ***P<0.001, ****P<0.0001 by unpaired t test.

FIG. 15C shows direct cell death effects (DCDE) induced by anti-hCD20 Abs. Raji cells (3.7×10⁵ cells in 570 L) seeded in the internal wells of 24-well plates in the presence of 10 μg/mL anti-hCD20 mAbs or Trastuzumab (isotype control). The plates were incubated at 37° C./5% CO₂ for 24 hours prior to analysis. Propidium iodide (PI) staining of non-viable cells. 500 μL mAb-treated cells were washed once with 200 μL DPBS/2% FBS and resuspended in 400 μL DPBS/2% FBS and stored on ice. 20 μL of propidium iodide solution (Invitrogen eBioscience) was added to the cell suspensions and mixed, and the PI-treated samples incubated on ice in the dark for 2-15 minutes prior to flow cytometric analysis in the phycoerythrin (PE) channel using a BD LSRFortessa X-20 instrument. At least 10,000 events were collected per analysis. Percentage of PI-positive cells were calculated as a proportion of the total cells after gating out debris based on forward/side scatter characteristics. Note that, in contrast to plots in (A) and (B) which report “healthy” cells, PI-positivity provides an inverse readout of “sick”/dead cells. Data are the mean±SD of triplicate measurements and are from one of two independent, representative experiments. *P<0.05; **P<0.01, ***P<0.001, ****P<0.0001 by unpaired t test.

FIG. 16 shows cross reactivity of anti-hCD20 Abs with mouse CD20. HEK293T cells transfected with either pCMV5-mCD20 (mCD20+; green) or pCMV5 (mCD20⁻; red) were incubated with 25 μg/mL Atto 488-labeled Abs followed by washing and flow-cytometric analysis. The identity of the Abs is indicated at the top. Anti-mCD20: commercially obtained anti-mCD20 Ab (eBioscience/Thermo Fisher). Data are from one of two independent, representative experiments.

FIG. 17 shows alignment of CD20 protein sequences from mice and humans. Grey shading: epitope of Ofatumumab. Yellow shading: epitope motif shared by Rituximab, Obinutuzumab (GA101), and Ocrelizumab (based on murine IgG2b Ab 2H7). Note: there is no chicken homolog for CD20 listed in the NCBI database.

FIG. 18A shows an illustrative scheme of the experimental outline for evaluation of in vivo efficacy of anti-hCD20 Abs. CB-17 SCID mice (4-6 mice/group) were injected intraperitoneally with 5×10⁵ CellTrace Violet-labelled Raji cells 16 hours prior to treatment with 1 ng (grey) or 10 ng (white) mAb or Dulbecco's phosphate-buffered saline (DPBS).

FIG. 18B shows representative box and whisker plots of intraperitoneal Raji cell counts following Ab treatment (median±interquartile range) and evaluation of in vivo efficacy of anti-hCD20 Abs. CB-17 SCID mice (4-6 mice/group) that were injected intraperitoneally with 5×10⁵ CellTrace Violet-labelled Raji cells 16 hours prior to treatment with 1 ng (grey) or 10 ng (white) mAb or Dulbecco's phosphate-buffered saline (DPBS). After 24 hours, the remaining Raji cells were flow cytometrically quantified as a measure of anti-tumor activity. To enable comparison of data generated in independent experiments, Raji cell counts were expressed as a percentage of the counts from the DPBS treatment group carried out on the same day. The prefix “ch” denotes Abs with a chicken variable region and “h” denotes a humanized variable region. *P<0.05, ***P<0.001, ****P<0.0001, ns, not significant (P>0.05), by two-way ANOVA followed by Dunnett's post-hoc analysis.

### DETAILED DESCRIPTION

Various embodiments of the invention are described herein as follows. In an illustrative aspect, a first antigen-binding molecule that specifically binds to CD20 is provided. The first antigen-binding molecule comprises an immunoglobulin heavy chain variable domain (VH) and an immunoglobulin light chain variable domain (VL), wherein the VH comprises a complementarity determining region 1 (VH CDR1) comprising the amino acid sequence of SEQ ID NO: 9, a VH CDR2 comprising the amino acid sequence of SEQ ID NO: 13 and a VH CDR3 comprising the amino acid sequence of SEQ ID NO: 17; and wherein the VL comprises a complementarity determining region 1 (VL CDR1) comprising the amino acid sequence of SEQ ID NO: 21, a VL CDR2 comprising the amino acid sequence of SEQ ID NO: 25, and a VL CDR3 comprising the amino acid sequence of SEQ ID NO: 29. The sequences include:

VH CDR1
(SEQ ID NO: 9)
SSYAM;
VH CDR2
(SEQ ID NO: 13)
EITNYAAGSGTWYGAAVK;
VH CDR3
(SEQ ID NO: 17)
KGPISGSIGYLSSIDA;
VL CDR1
(SEQ ID NO: 21)
SGGSSRYGYG;
VL CDR2
(SEQ ID NO: 25)
WNDKRPS;
and
VL CDR3
(SEQ ID NO: 29)
GNYDNNDPA.

In an embodiment, the VH comprises an amino acid sequence having at least 80% sequence identity to SEQ ID NO: 1. In an embodiment, the VH comprises an amino acid sequence having at least 85% sequence identity to SEQ ID NO: 1. In an embodiment, the VH comprises an amino acid sequence having at least 90% sequence identity to SEQ ID NO: 1. In an embodiment, the VH comprises an amino acid sequence having at least 91% sequence identity to SEQ ID NO: 1. In an embodiment, the VH comprises an amino acid sequence having at least 92% sequence identity to SEQ ID NO: 1. In an embodiment, the VH comprises an amino acid sequence having at least 93% sequence identity to SEQ ID NO: 1. In an embodiment, the VH comprises an amino acid sequence having at least 94% sequence identity to SEQ ID NO: 1. In an embodiment, the VH comprises an amino acid sequence having at least 95% sequence identity to SEQ ID NO: 1. In an embodiment, the VH comprises an amino acid sequence having at least 96% sequence identity to SEQ ID NO: 1. In an embodiment, the VH comprises an amino acid sequence having at least 97% sequence identity to SEQ ID NO: 1. In an embodiment, the VH comprises an amino acid sequence having at least 98% sequence identity to SEQ ID NO: 1. In an embodiment, the VH comprises an amino acid sequence having at least 99% sequence identity to SEQ ID NO: 1.

In an embodiment, the VL comprises an amino acid sequence having at least 80% sequence identity to SEQ ID NO: 5. In an embodiment, the VL comprises an amino acid sequence having at least 85% sequence identity to SEQ ID NO: 5. In an embodiment, the VL comprises an amino acid sequence having at least 90% sequence identity to SEQ ID NO: 5. In an embodiment, the VL comprises an amino acid sequence having at least 91% sequence identity to SEQ ID NO: 5. In an embodiment, the VL comprises an amino acid sequence having at least 92% sequence identity to SEQ ID NO: 5. In an embodiment, the VL comprises an amino acid sequence having at least 93% sequence identity to SEQ ID NO: 5. In an embodiment, the VL comprises an amino acid sequence having at least 94% sequence identity to SEQ ID NO: 5. In an embodiment, the VL comprises an amino acid sequence having at least 95% sequence identity to SEQ ID NO: 5. In an embodiment, the VL comprises an amino acid sequence having at least 96% sequence identity to SEQ ID NO: 5. In an embodiment, the VL comprises an amino acid sequence having at least 97% sequence identity to SEQ ID NO: 5. In an embodiment, the VL comprises an amino acid sequence having at least 98% sequence identity to SEQ ID NO: 5. In an embodiment, the VL comprises an amino acid sequence having at least 99% sequence identity to SEQ ID NO: 5.

In an illustrative aspect, a second antigen-binding molecule that specifically binds to CD20 is provided. The second antigen-binding molecule comprises an immunoglobulin heavy chain variable domain (VH) and an immunoglobulin light chain variable domain (VL), wherein the VH comprises a complementarity determining region 1 (VH CDR1) comprising the amino acid sequence of SEQ ID NO: 10, a VH CDR2 comprising the amino acid sequence of SEQ ID NO: 14 and a VH CDR3 comprising the amino acid sequence of SEQ ID NO: 18; and wherein the VL comprises a complementarity determining region 1 (VL CDR1) comprising the amino acid sequence of SEQ ID NO: 22, a VL CDR2 comprising the amino acid sequence of SEQ ID NO: 26, and a VL CDR3 comprising the amino acid sequence of SEQ ID NO: 30:

VH CDR1
(SEQ ID NO: 10)
SSSYIN;
VH CDR2
(SEQ ID NO: 14)
QINKDGGGSWYAPVVK;
VH CDR3
(SEQ ID NO: 18)
KNADSGCVGVGGCIDT;
VL CDR1
(SEQ ID NO: 22)
SGGSYYYGGNYYYG;
VL CDR2
(SEQ ID NO: 26)
NNNKRPS;
and
VL CDR3
(SEQ ID NO: 30)
GGYDSSYVAI.

In an embodiment, the VH comprises an amino acid sequence having at least 80% sequence identity to SEQ ID NO: 2. In an embodiment, the VH comprises an amino acid sequence having at least 85% sequence identity to SEQ ID NO: 2. In an embodiment, the VH comprises an amino acid sequence having at least 90% sequence identity to SEQ ID NO: 2. In an embodiment, the VH comprises an amino acid sequence having at least 91% sequence identity to SEQ ID NO: 2. In an embodiment, the VH comprises an amino acid sequence having at least 92% sequence identity to SEQ ID NO: 2. In an embodiment, the VH comprises an amino acid sequence having at least 93% sequence identity to SEQ ID NO: 2. In an embodiment, the VH comprises an amino acid sequence having at least 94% sequence identity to SEQ ID NO: 2. In an embodiment, the VH comprises an amino acid sequence having at least 95% sequence identity to SEQ ID NO: 2. In an embodiment, the VH comprises an amino acid sequence having at least 96% sequence identity to SEQ ID NO: 2. In an embodiment, the VH comprises an amino acid sequence having at least 97% sequence identity to SEQ ID NO: 2. In an embodiment, the VH comprises an amino acid sequence having at least 98% sequence identity to SEQ ID NO: 2. In an embodiment, the VH comprises an amino acid sequence having at least 99% sequence identity to SEQ ID NO: 2.

In an embodiment, the VL comprises an amino acid sequence having at least 80% sequence identity to SEQ ID NO: 6. In an embodiment, the VL comprises an amino acid sequence having at least 85% sequence identity to SEQ ID NO: 6. In an embodiment, the VL comprises an amino acid sequence having at least 90% sequence identity to SEQ ID NO: 6. In an embodiment, the VL comprises an amino acid sequence having at least 91% sequence identity to SEQ ID NO: 6. In an embodiment, the VL comprises an amino acid sequence having at least 92% sequence identity to SEQ ID NO: 6. In an embodiment, the VL comprises an amino acid sequence having at least 93% sequence identity to SEQ ID NO: 6. In an embodiment, the VL comprises an amino acid sequence having at least 94% sequence identity to SEQ ID NO: 6. In an embodiment, the VL comprises an amino acid sequence having at least 95% sequence identity to SEQ ID NO: 6. In an embodiment, the VL comprises an amino acid sequence having at least 96% sequence identity to SEQ ID NO: 6. In an embodiment, the VL comprises an amino acid sequence having at least 97% sequence identity to SEQ ID NO: 6. In an embodiment, the VL comprises an amino acid sequence having at least 98% sequence identity to SEQ ID NO: 6. In an embodiment, the VL comprises an amino acid sequence having at least 99% sequence identity to SEQ ID NO: 6.

In an embodiment, the VH comprises an amino acid sequence having at least 80% sequence identity to SEQ ID NO: 33. In an embodiment, the VH comprises an amino acid sequence having at least 85% sequence identity to SEQ ID NO: 33. In an embodiment, the VH comprises an amino acid sequence having at least 90% sequence identity to SEQ ID NO: 33. In an embodiment, the VH comprises an amino acid sequence having at least 91% sequence identity to SEQ ID NO: 33. In an embodiment, the VH comprises an amino acid sequence having at least 92% sequence identity to SEQ ID NO: 33. In an embodiment, the VH comprises an amino acid sequence having at least 93% sequence identity to SEQ ID NO: 33. In an embodiment, the VH comprises an amino acid sequence having at least 94% sequence identity to SEQ ID NO: 33. In an embodiment, the VH comprises an amino acid sequence having at least 95% sequence identity to SEQ ID NO: 33. In an embodiment, the VH comprises an amino acid sequence having at least 96% sequence identity to SEQ ID NO: 33. In an embodiment, the VH comprises an amino acid sequence having at least 97% sequence identity to SEQ ID NO: 33. In an embodiment, the VH comprises an amino acid sequence having at least 98% sequence identity to SEQ ID NO: 33. In an embodiment, the VH comprises an amino acid sequence having at least 99% sequence identity to SEQ ID NO: 33.

In an embodiment, the VL comprises an amino acid sequence having at least 80% sequence identity to SEQ ID NO: 34. In an embodiment, the VL comprises an amino acid sequence having at least 85% sequence identity to SEQ ID NO: 34. In an embodiment, the VL comprises an amino acid sequence having at least 90% sequence identity to SEQ ID NO: 34. In an embodiment, the VL comprises an amino acid sequence having at least 91% sequence identity to SEQ ID NO: 34. In an embodiment, the VL comprises an amino acid sequence having at least 92% sequence identity to SEQ ID NO: 34. In an embodiment, the VL comprises an amino acid sequence having at least 93% sequence identity to SEQ ID NO: 34. In an embodiment, the VL comprises an amino acid sequence having at least 94% sequence identity to SEQ ID NO: 34. In an embodiment, the VL comprises an amino acid sequence having at least 95% sequence identity to SEQ ID NO: 34. In an embodiment, the VL comprises an amino acid sequence having at least 96% sequence identity to SEQ ID NO: 34. In an embodiment, the VL comprises an amino acid sequence having at least 97% sequence identity to SEQ ID NO: 34. In an embodiment, the VL comprises an amino acid sequence having at least 98% sequence identity to SEQ ID NO: 34. In an embodiment, the VL comprises an amino acid sequence having at least 99% sequence identity to SEQ ID NO: 34.

In an illustrative aspect, a third antigen-binding molecule that specifically binds to CD20 is provided. The third antigen-binding molecule comprises an immunoglobulin heavy chain variable domain (VH) and an immunoglobulin light chain variable domain (VL), wherein the VH comprises a complementarity determining region 1 (VH CDR1) comprising the amino acid sequence of SEQ ID NO: 11, a VH CDR2 comprising the amino acid sequence of SEQ ID NO: 15 and a VH CDR3 comprising the amino acid sequence of SEQ ID NO: 19; and wherein the VL comprises a complementarity determining region 1 (VL CDR1) comprising the amino acid sequence of SEQ ID NO: 23, a VL CDR2 comprising the amino acid sequence of SEQ ID NO: 27, and a VL CDR3 comprising the amino acid sequence of SEQ ID NO: 31:

VH CDR1
(SEQ ID NO: 11)
NSNGMG;
VH CDR2
(SEQ ID NO: 15)
GIHSSGRYTYYGTAVK;
VH CDR3
(SEQ ID NO: 19)
KNADSAYGYWYAGSIDA;
VL CDR1
(SEQ ID NO: 23)
SGGYSSYGYS;
VL CDR2
(SEQ ID NO: 27)
NNNNRPS;
and
VL CDR3
(SEQ ID NO: 31)
AFTDYSSLAGV.

In an embodiment, the VH comprises an amino acid sequence having at least 80% sequence identity to SEQ ID NO: 3. In an embodiment, the VH comprises an amino acid sequence having at least 85% sequence identity to SEQ ID NO: 3. In an embodiment, the VH comprises an amino acid sequence having at least 90% sequence identity to SEQ ID NO: 3. In an embodiment, the VH comprises an amino acid sequence having at least 91% sequence identity to SEQ ID NO: 3. In an embodiment, the VH comprises an amino acid sequence having at least 92% sequence identity to SEQ ID NO: 3. In an embodiment, the VH comprises an amino acid sequence having at least 93% sequence identity to SEQ ID NO: 3. In an embodiment, the VH comprises an amino acid sequence having at least 94% sequence identity to SEQ ID NO: 3. In an embodiment, the VH comprises an amino acid sequence having at least 95% sequence identity to SEQ ID NO: 3. In an embodiment, the VH comprises an amino acid sequence having at least 96% sequence identity to SEQ ID NO: 3. In an embodiment, the VH comprises an amino acid sequence having at least 97% sequence identity to SEQ ID NO: 3. In an embodiment, the VH comprises an amino acid sequence having at least 98% sequence identity to SEQ ID NO: 3. In an embodiment, the VH comprises an amino acid sequence having at least 99% sequence identity to SEQ ID NO: 3.

In an embodiment, the VL comprises an amino acid sequence having at least 80% sequence identity to SEQ ID NO: 7. In an embodiment, the VL comprises an amino acid sequence having at least 85% sequence identity to SEQ ID NO: 7. In an embodiment, the VL comprises an amino acid sequence having at least 90% sequence identity to SEQ ID NO: 7. In an embodiment, the VL comprises an amino acid sequence having at least 91% sequence identity to SEQ ID NO: 7. In an embodiment, the VL comprises an amino acid sequence having at least 92% sequence identity to SEQ ID NO: 7. In an embodiment, the VL comprises an amino acid sequence having at least 93% sequence identity to SEQ ID NO: 7. In an embodiment, the VL comprises an amino acid sequence having at least 94% sequence identity to SEQ ID NO: 7. In an embodiment, the VL comprises an amino acid sequence having at least 95% sequence identity to SEQ ID NO: 7. In an embodiment, the VL comprises an amino acid sequence having at least 96% sequence identity to SEQ ID NO: 7. In an embodiment, the VL comprises an amino acid sequence having at least 97% sequence identity to SEQ ID NO: 7. In an embodiment, the VL comprises an amino acid sequence having at least 98% sequence identity to SEQ ID NO: 7. In an embodiment, the VL comprises an amino acid sequence having at least 99% sequence identity to SEQ ID NO: 7.

In an illustrative aspect, a fourth antigen-binding molecule that specifically binds to CD20 is provided. The fourth antigen-binding molecule comprises an immunoglobulin heavy chain variable domain (VH) and an immunoglobulin light chain variable domain (VL), wherein the VH comprises a complementarity determining region 1 (VH CDR1) comprising the amino acid sequence of SEQ ID NO: 12, a VH CDR2 comprising the amino acid sequence of SEQ ID NO: 16 and a VH CDR3 comprising the amino acid sequence of SEQ ID NO: 20; and wherein the VL comprises a complementarity determining region 1 (VL CDR1) comprising the amino acid sequence of SEQ ID NO: 24, a VL CDR2 comprising the amino acid sequence of SEQ ID NO: 28, and a VL CDR3 comprising the amino acid sequence of SEQ ID NO: 32:

VH CDR1
(SEQ ID NO: 12)
DRHGMF;
VH CDR2
(SEQ ID NO: 16)
GIRSTGSYPKYGPAVK;
VH CDR3
(SEQ ID NO: 20)
RNVYIGDYDGDSIDA;
VL CDR1
(SEQ ID NO: 24)
SGSSSGYGSYYG;
VL CDR2
(SEQ ID NO: 28)
ENTNRPS;
and
VL CDR3
(SEQ ID NO: 32)
GSADDSGNSAI.

In an embodiment, the VH comprises an amino acid sequence having at least 80% sequence identity to SEQ ID NO: 4. In an embodiment, the VH comprises an amino acid sequence having at least 85% sequence identity to SEQ ID NO: 4. In an embodiment, the VH comprises an amino acid sequence having at least 90% sequence identity to SEQ ID NO: 4. In an embodiment, the VH comprises an amino acid sequence having at least 91% sequence identity to SEQ ID NO: 4. In an embodiment, the VH comprises an amino acid sequence having at least 92% sequence identity to SEQ ID NO: 4. In an embodiment, the VH comprises an amino acid sequence having at least 93% sequence identity to SEQ ID NO: 4. In an embodiment, the VH comprises an amino acid sequence having at least 94% sequence identity to SEQ ID NO: 4. In an embodiment, the VH comprises an amino acid sequence having at least 95% sequence identity to SEQ ID NO: 4. In an embodiment, the VH comprises an amino acid sequence having at least 96% sequence identity to SEQ ID NO: 4. In an embodiment, the VH comprises an amino acid sequence having at least 97% sequence identity to SEQ ID NO: 4. In an embodiment, the VH comprises an amino acid sequence having at least 98% sequence identity to SEQ ID NO: 4. In an embodiment, the VH comprises an amino acid sequence having at least 99% sequence identity to SEQ ID NO: 4.

In an embodiment, the VL comprises an amino acid sequence having at least 80% sequence identity to SEQ ID NO: 8. In an embodiment, the VL comprises an amino acid sequence having at least 85% sequence identity to SEQ ID NO: 8. In an embodiment, the VL comprises an amino acid sequence having at least 90% sequence identity to SEQ ID NO: 8. In an embodiment, the VL comprises an amino acid sequence having at least 91% sequence identity to SEQ ID NO: 8. In an embodiment, the VL comprises an amino acid sequence having at least 92% sequence identity to SEQ ID NO: 8. In an embodiment, the VL comprises an amino acid sequence having at least 93% sequence identity to SEQ ID NO: 8. In an embodiment, the VL comprises an amino acid sequence having at least 94% sequence identity to SEQ ID NO: 8. In an embodiment, the VL comprises an amino acid sequence having at least 95% sequence identity to SEQ ID NO: 8. In an embodiment, the VL comprises an amino acid sequence having at least 96% sequence identity to SEQ ID NO: 8. In an embodiment, the VL comprises an amino acid sequence having at least 97% sequence identity to SEQ ID NO: 8. In an embodiment, the VL comprises an amino acid sequence having at least 98% sequence identity to SEQ ID NO: 8. In an embodiment, the VL comprises an amino acid sequence having at least 99% sequence identity to SEQ ID NO: 8.

In an embodiment, the antigen-binding molecule is an antibody or a CD20-binding fragment thereof. In an embodiment, the CD20-binding fragment is selected from the group consisting of an Fab fragment, an scFab, an Fab′, a single chain variable fragment (scFv) and a one-armed antibody. In an embodiment, the antigen-binding molecule is a humanized antibody or CD20-binding fragment thereof.

In an illustrative aspect, an isolated nucleic acid molecule is provided. The isolated nucleic acid comprises a nucleic acid sequence encoding any of the antigen-binding molecules described herein.

In an illustrative aspect, an expression construct is provided. The expression construct comprises a nucleic acid sequence encoding the antigen-binding molecule of any of the antigen-binding molecules described herein, operably linked to one or more regulatory sequences. In an illustrative aspect, a host cell comprising the expression construct is provided.

In an illustrative aspect, a vector comprising a nucleic acid sequence encoding any of the antigen-binding molecules described herein is provided.

In an illustrative aspect, a pharmaceutical composition is provided. The pharmaceutical composition comprises any of the antigen-binding molecules described herein and a pharmaceutically acceptable carrier.

In an illustrative aspect, a method of treating a disease in a patient is provided. The method comprises administering to the patient in need thereof any of the antigen-binding molecules, or a CD20-binding fragment thereof, the vector, or the pharmaceutical composition as described herein. In any embodiment herein, the administration can be made in a therapeutically effective amount of any of the antigen-binding molecules, or a CD20-binding fragment thereof, the vector, or the pharmaceutical composition as described herein.

In an embodiment, the disease is a cancer. In an embodiment, the cancer is a CD20 positive B-cell cancer. In an embodiment, the disease is a blood cancer. In an embodiment, the blood cancer is a CD20 positive B-cell blood cancer. In an embodiment, the disease is Non-Hodgkin's Lymphoma (NHL). In an embodiment, the disease is Hodgkin's Lymphoma (HL). In an embodiment, the disease is CD20 positive B-cell NHL. In an embodiment, the disease is Chronic Lymphocytic Leukemia (CLL). In an embodiment, the disease is Rheumatoid Arthritis. In an embodiment, the disease is Microscopic Polyangiitis (MP). In an embodiment, the disease is Granulomatosis with Polyangiitis (GPA). In an embodiment, the disease is Pemphigus Vulgaris (PV).

In an illustrative aspect, a method of treating or preventing a cancer induced to proliferate by CD20 and conditions associated therewith is provided. The method comprises administering to a subject in need thereof any of the antigen-binding molecules, or a CD20-binding fragment thereof, the vector, or the pharmaceutical composition as described herein. In any embodiment herein, the administration can be made in a therapeutically effective amount of any of the antigen-binding molecules, or a CD20-binding fragment thereof, the vector, or the pharmaceutical composition as described herein.

In an embodiment, the cancer is a CD20 positive B-cell cancer. In an embodiment, the cancer is a blood cancer. In an embodiment, the blood cancer is a CD20 positive B-cell blood cancer. In an embodiment, the cancer is a lymphoma. In an embodiment, the lymphoma is a CD20 positive B-cell lymphoma.

In an illustrative aspect, a kit comprising any of the antigen-binding molecules, or a CD20-binding fragment thereof, the vector, or the pharmaceutical composition as described herein is provided.

As used herein, an “antigen-binding molecule” refers to a molecule that has binding affinity for a target antigen. It will be understood that this term extends to immunoglobulins, immunoglobulin fragments and non-immunoglobulin-derived protein frameworks that exhibit antigen-binding activity. Illustrative examples of suitable antigen-binding molecules include antibodies and antigen-binding fragments thereof. Preferably, the antigen-binding molecule binds specifically to CD20 so as to redce its activity. The antigen-binding molecule, as described herein, can be conjugated to another molecule or moiety, including functional moieties (e.g., toxins), detectable moieties (e.g., fluorescent molecules, radioisotopes), small molecule drugs and polypeptides.

As used herein, the term “antibody” refers to any antigen-binding molecule or molecular complex comprising at least one complementarity determining region (CDR) that binds specifically to, or interacts specifically with, the target antigen. The term “antibody” includes full-length immunoglobulin molecules comprising two heavy (H) chains and two light (L) chains inter-connected by disulfide bonds, as well as multimers thereof (e.g., TgM). Each heavy chain comprises a heavy chain variable region (which may be abbreviated as HCVR, VH or VH) and a heavy chain constant region. The heavy chain constant region typically comprises three domains—CH1, CH2 and CH3. Each light chain comprises a light chain variable region (which may be abbreviated as LCVR, VL, VK, VK or VL) and a light chain constant region. The light chain constant region will typically comprise one domain (CL1). The VH and VL regions can be further subdivided into regions of hypervariability, termed complementarity determining regions (CDRs), interspersed with regions that are more conserved, also referred to as framework regions (FR).

Suitable antibodies include antibodies of any class, such as IgG, IgA, or IgM (including sub-classes thereof). There are five major classes of immunoglobulins: IgA, IgD, IgE, IgG, and IgM, characterised by heavy-chain constant regions α, δ, ε, γ, and μ, respectively. Several antibody classes may be further divided into subclasses (isotypes), e.g., IgG1, IgG2, TgG3, IgG4, IgA1 and TgA2. The subunit structures and three-dimensional configurations of different classes of immunoglobulins will be well known to persons skilled in the art.

As used herein, the term “complementarity determining region” (CDR) refers to the region of an immunoglobulin variable domain that recognizes and binds to the target antigen. Each variable domain may comprises up to three CDR sequences, identified as CDR1, CDR2 and CDR3. The amino acid sequence of each CDR is often defined by Rabat numbering (see Rabat et al, Sequences of Proteins of Immunological Interest, 5th Ed. Public Health Service, National Institutes of Health, Bethesda, Md. (1991)) and/or by Chothia numbering (see Chothia and Lesk J. Mol. Biol. 196:901-917 (1987)).

The terms “antigen-binding fragment”, “antigen-binding portion”, “antigen-binding domain”, “antigen-binding site” and the like are used interchangeably herein to refer to a part of an antigen-binding molecule that retains the ability to bind to the target antigen, for instance CD20. These terms include naturally occurring, enzymatically obtainable, synthetic or genetically engineered (recombinant) polypeptides and glycoproteins that specifically bind to CD20 to form a complex.

Antigen-binding fragments may be derived, for example, from naturally-derived immunoglobulin molecules using any suitable method known to persons skilled in the art, illustrative examples of which include proteolytic digestion or recombinant genetic engineering techniques involving the manipulation and expression of nucleic acid sequences encoding antibody variable and optionally constant domains. Suitable nucleic acid sequences are known and/or are readily available from, e.g., commercial sources, DNA libraries (including, e.g, phage-antibody libraries), or can be synthesized. The nucleic acid sequences may be sequenced and manipulated chemically or by using molecular biology techniques, for example, to arrange one or more variable and/or constant domains into a suitable configuration, or to introduce codons, create cysteine residues, modify, add or delete amino acids, etc.

Non-limiting examples of suitable antigen-binding fragments include: (i) Fab fragments; (ii) F(ab′)2 fragments; (iii) Fd fragments; (iv) Fv fragments; (v) single-chain Fv (scFv) molecules; (vi) dAb fragments; and (vii) minimal recognition units consisting of the amino acid residues that mimic the hypervariable region of an antibody (e.g, an isolated CDR such as a CDR3 peptide), or a constrained FR3-CDR3-FR4 peptide. Other engineered molecules, such as domain-specific antibodies, single domain antibodies, domain-deleted antibodies, chimeric antibodies, CDR-grafted antibodies, one-armed antibodies, diabodies, triabodies, tetrabodies, minibodies, nanobodies (e.g. monovalent nanobodies, bivalent nanobodies, etc.), and small modular immunopharmaceuticals (SMIPs), are also encompassed by the term “antigen-binding fragment,” as used herein.

In an embodiment, an antigen-binding fragment comprises at least one immunoglobulin variable domain. The variable domain may comprise an amino acid sequence of any suitable length or composition and will generally comprise at least one CDR which is adjacent to or in frame with one or more framework sequences. Where the antigen-binding fragment comprises a VH domain and a VL domain, the VH and VL domains may be situated relative to one another in any suitable arrangement. For example, the variable region may be dimeric and contain VH-VH, VH-VL or VL-VL dimers. Alternatively, the antigen-binding fragment of an antibody may contain a monomeric VH or VL domain.

In some embodiments, an antigen-binding fragment may comprise at least one variable domain covalently linked to at least one constant domain. Non-limiting configurations of variable and constant domains that may be found within an antigen-binding fragment include: (i) VH-CH1; (ii) VH-CH2; (iii) VH-CH3; (iv) VH-CH1-CH2; (V) VH-CH1-CH2-CH3, (vi) VH-CH2-CH3; (vii) VH-CL; (viii) VL-CH1; (ix) VL-CH2, (X) VL-CH3; (xi) VL-CH1-CH2; (xii) VL-CH1-CH2-CH3; (xiii) VL-CH2-CH3; and (xiv) VL-CL. In any configuration of variable and constant domains, including any of the exemplary configurations listed above, the variable and constant domains may be either directly linked to one another or may be linked by a full or partial hinge or linker region. A hinge region may consist of at least 2 (e.g., 5, 10, 15, 20, 40, 60 or more) amino acids which result in a flexible or semi-flexible linkage between adjacent variable and/or constant domains in a single polypeptide molecule. In some embodiments, the antigen-binding fragment, as herein described, may comprise a homo-dimer or hetero-dimer (or other multimer) of any of the variable and constant domain configurations listed above in non-covalent association with one another and/or with one or more monomeric VH or VL domains (e.g, by disulfide bond(s)). A multispecific antigen-binding molecule will typically comprise at least two different variable domains, wherein each variable domain is capable of specifically binding to a separate antigen or to a different epitope on the same antigen. Any multispecific antigen-binding molecule format, including bispecific antigen-binding molecule formats, may be adapted for use in the context of an antigen-binding fragment of an antibody of the present disclosure using routine techniques available in the art.

The term “variable region” or “variable domain” refers to the domain of an immunoglobulin heavy or light chain that is involved in binding to the target antigen. The variable domains of the heavy chain and tight chain (VH and VL, respectively) of a native immunoglobulin molecule will generally have similar structures, with each domain comprising four conserved framework regions and three hypervariable regions (HVRs). See, e.g., Kindt etal., Kuby Immunology, 6th ed., W.H. Freeman and Co., page 91 (2007). A single VH or VL domain may be sufficient to confer antigen-binding specificity.

As used herein, the term “humanized” means that the antigen-binding molecule comprises an amino acid sequence that is compatible with humans, such that the amino acid sequence is unlikely to be seen as foreign by the immune system of a human subject. In an embodiment, the humanized antigen-binding molecule comprises one or more immunoglobulin framework regions derived from one or more human immunoglobulin molecules. In some embodiments, all of the framework regions of the humanized antigen-binding molecule will be derived from one or more human immunoglobulin molecules. The humanized antibody may optionally comprise an immunoglobulin heavy chain constant region derived from a human immunoglobulin molecule.

The phrase “specifically binds” or “specific binding” refers to a binding reaction between two molecules that is at least two times the background and more typically more than 10 to 100 times background molecular associations under physiological conditions. When using one or more detectable binding agents that are proteins, specific binding is determinative of the presence of the protein, in a heterogeneous population of proteins and other biologies. Thus, under designated immunoassay conditions, the specified antigen-binding molecule binds to a particular antigenic determinant, thereby identifying its presence. Specific binding to an antigenic determinant under such conditions requires an antigen-binding molecule that is selected for its specificity to that determinant. This selection may be achieved by subtracting out antigen-binding molecules that cross-react with other molecules. A variety of immunoassay formats may be used to select antigen-binding molecules (e.g., immunoglobulins) [such that they are specifically immunoreactive with a particular antigen. For example, solid-phase ELISA immunoassays are routinely used to select antibodies specifically immunoreactive with a protein (see, e.g., Harlow & Lane, Antibodies, A Laboratory Manual (1988) for a description of immunoassay formats and conditions that can be used to determine specific immunoreactivity). Methods of determining binding affinity and specificity are also well known in the art (see, for example, Harlow and Lane, supra); Friefelder, “Physical Biochemistry: Applications to biochemistry and molecular biology” (W.H. Freeman and Co. 1976))

“Affinity” or “binding affinity” refers to the strength of the sum total of non-covalent interactions between a single binding site of a molecule (e.g, an antigen-binding molecule) and its binding partner (e.g, an antigen). Unless indicated otherwise, as used herein, “binding affinity” refers to intrinsic binding affinity which reflects a 1:1 interaction between members of a binding pair e.g, an antigen-binding molecule. The affinity of a molecule X for its partner Y can generally be represented by the dissociation constant (Kd), which is the ratio of dissociation and association rate constants (koff and kon, respectively). Thus, equivalent affinities may comprise different rate constants, as long as the ratio of the rate constants remains the same. Affinity can be measured by common methods known in the art, including those described herein. A particular method for measuring affinity is Surface Plasmon Resonance (SPR).

As used herein, the term “modified antibody” includes synthetic forms of antibodies which are altered such that they are not naturally occurring, e.g., antibodies that comprise at least two heavy chain portions but not two complete heavy chains (such as domain deleted antibodies or minibodies); multispecific forms of antibodies (e.g., bispecific, trispecific, etc.) altered to bind to two or more different antigens or to different epitopes on a single antigen; heavy chain molecules joined to scFv molecules and the like. ScFv molecules are known in the art and are described, e.g., in U.S. Pat. No. 5,892,019. In addition, the term “modified antibody” includes multivalent forms of antibodies (e.g., bivalent, tetravalent, etc., antibodies that bind to three or more copies of the same antigen).

Also disclosed herein is a vector that comprises a nucleic acid encoding the CD20-binding molecules, as described herein. By “vector” is meant a nucleic acid molecule, preferably a DNA molecule derived, for example, from a plasmid, bacteriophage, or vims, into which a nucleic acid sequence may be inserted or cloned. A vector preferably contains one or more unique restriction sites and may be capable of autonomous replication in a defined host cell including a target cell or tissue or a progenitor cell or tissue thereof, or be integrable with the genome of the defined host such that the cloned sequence is reproducible. Accordingly, the vector may be an autonomously replicating vector, i.e., a vector that exists as an extrachromosomal entity, the replication of which is independent of chromosomal replication, e.g., a linear or closed circular plasmid, an extrachromosomal element, a minichromosome, or an artificial chromosome. The vector may contain any means for assuring self-replication. Alternatively, the vector may be one which, when introduced into the host cell, is integrated into the genome and replicated together with the chromosome(s) into which it has been integrated. A vector system may comprise a single vector or plasmid, two or more vectors or plasmids, which together contain the total DNA to be introduced into the genome of the host cell, or atransposon. The choice of the vector will typically depend on the compatibility of the vector with the host cell into which the vector is to be introduced. The vector may also include a selection marker such as an antibiotic resistance gene that can be used for selection of suitable transformants. Examples of such resistance genes are well known to those of skill in the art.

The term “construct” refers to a recombinant genetic molecule including one or more isolated nucleic acid sequences from different sources. Thus, constructs are chimeric molecules in which two or more nucleic acid sequences of different origin are assembled into a single nucleic add molecule and include any construct that contains (1) nucleic acid sequences, including regulatory and coding sequences that are not found together in nature (i.e., at least one of the nucleotide sequences is heterologous with respect to at least one of its other nucleotide sequences), or (2) sequences encoding parts of functional RNA molecules or proteins not naturally adjoined, or (3) parts of promoters that are not naturally adjoined. Representative constructs include any recombinant nucleic acid molecule such as a plasmid, cosmid, virus, autonomously replicating polynucleotide molecule, phage, or linear or circular single stranded or double stranded DNA or RNA nucleic acid molecule, derived from any source, capable of genomic integration or autonomous replication, comprising a nucleic acid molecule where one or more nucleic acid molecules have been operably linked. Constructs of the present invention will generally include the necessary elements to direct expression of a nucleic acid sequence of interest that is also contained in the construct, such as, for example, a target nucleic acid sequence or a modulator nucleic acid sequence. Such elements may include control elements or regulatory sequences such as a promoter that is operably linked to (so as to direct transcription of) the nucleic acid sequence of interest, and often includes a polyadenylation sequence as well. Within certain embodiments of the invention, the construct may be contained within a vector. In addition to the components of the construct, the vector may include, for example, one or more selectable markers, one or more origins of replication, such as prokaryotic and eukaryotic origins, at least one multiple cloning site, and/or elements to facilitate stable integration of the construct into the genome of a host cell. Two or more constructs can be contained within a single nucleic acid molecule, such as a single vector, or can be contained within two or more separate nucleic acid molecules, such as two or more separate vectors. An “expression construct” generally includes at least a control sequence operably linked to a nucleotide sequence of interest. In this manner, for example, promoters in operable connection with the nucleotide sequences to be expressed are provided in expression constructs for expression in an organism or part thereof including a host cell. For the practice of the present invention, conventional compositions and methods for preparing and using constructs and host cells are well known to one skilled in the art, see for example, Molecular Cloning: A Laboratory Manual, 3rd edition Volumes 1, 2, and 3. J. F. Sambrook, D. W. Russell, and N. Irwin, Cold Spring Harbor Laboratory Press, 2000.

By “control element”, “control sequence”, “regulatory sequence” and the like, as used herein, is meant a nucleic acid sequence (e.g., DNA) necessary for expression of an operably linked coding sequence in a particular host cell. The control sequences that are suitable for prokaryotic cells for example, include a promoter, and optionally a cis-acting sequence such as an operator sequence and a ribosome binding site. Control sequences that are suitable for eukaryotic cells include transcriptional control sequences such as promoters, polyadenylation signals, transcriptional enhancers, translational control sequences such as translational enhancers and internal ribosome binding sites (IRES), nucleic acid sequences that modulate mRNA stability, as well as targeting sequences that target a product encoded by a transcribed polynucleotide to an intracellular compartment within a cell or to the extracellular environment.

Also disclosed herein is a host cell comprising the construct as defined herein. The terms “host”, ‘host cell”, ‘host cell line” and ‘host cell culture” are used interchangeably and refer to cells into which exogenous nucleic acid has been introduced, including the progeny of such cells. Host cells include “transformants” and “transformed cells”, which include the primary transformed cell and progeny derived therefrom without regard to the number of passages. Progeny may not be completely identical in nucleic acid content to a parent cell, but may contain mutations. Mutant progeny that have the same function or biological activity as screened or selected for in the originally transformed cell are included herein. A host cell is any type of cellular system that can be used to generate the antigen binding molecules of the present invention.

Also disclosed herein is a pharmaceutical composition comprising the CD20-binding molecule or a vector, as described herein, and a pharmaceutically acceptable carrier. By “pharmaceutically acceptable carrier” is meant a pharmaceutical vehicle comprised of a material that is not biologically or otherwise undesirable, i.e., the material may be administered to a subject along with the selected active agent without causing any or a substantial adverse reaction. Carriers may include excipients and other additives such as diluents, detergents, coloring agents, wetting or emulsifying agents, pH buffering agents, preservatives, and the like.

Representative pharmaceutically acceptable carriers include any and all solvents, dispersion media, coatings, surfactants, antioxidants, preservatives (e.g, antibacterial agents, antifungal agents), isotonic agents, absorption delaying agents, salts, preservatives, drugs, drug stabilizers, gels, binders, excipients, disintegration agents, lubricants, sweetening agents, flavoring agents, dyes, such like materials and combinations thereof, as would be known to one of ordinary skill in the art (see, for example, Remington's Pharmaceutical Sciences, 18th Ed. Mack Printing Company, 1990, pp. 1289-1329, incorporated herein by reference). Except insofar as any conventional carrier is incompatible with the active ingredient(s), its use in the pharmaceutical compositions is contemplated.

The pharmaceutical compositions may be in a variety of forms. These include, for example, liquid, semi-solid and solid dosage forms, such as liquid solutions (e.g., injectable and infusible solutions), dispersions or suspensions, liposomes and suppositories. The preferred form depends on the intended mode of administration and therapeutic application. Suitable pharmaceutical compositions may be administered intravenously, subcutaneously or intramuscularly. In some embodiments, the compositions are in the form of injectable or infusible solutions. A preferred mode of administration is parenteral (e.g., intravenous, subcutaneous, intraperitoneal, intramuscular). In specific embodiments, the pharmaceutical composition is administered by intravenous infusion or injection. In other embodiments, the pharmaceutical composition is administered by intramuscular or subcutaneous injection.

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural and intrastemal injection and infusion.

Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. In the subject invention, pharmaceutically acceptable carriers include, but are not limited to, 0.01-0.1M and preferably 0.05M phosphate buffer or 0.8% saline. Other common parenteral vehicles include sodium phosphate solutions, Ringer's dextrose, dextrose and sodium chloride, lactated Ringer's, or fixed oils. Intravenous vehicles include fluid and nutrient replenishers, electrolyte replenishers, such as those based on Ringer's dextrose, and the like. Preservatives and other additives can also be present such as for example, antimicrobials, antioxidants, chelating agents, and inert gases and the like.

More particularly, pharmaceutical compositions suitable for injectable use include sterile aqueous solutions (where water soluble) or dispersions and sterile powders for the extemporaneous preparation of sterile injectable solutions or dispersions. In such cases, the composition must be sterile and should be fluid to the extent that easy syringability exists. It should be stable under the conditions of manufacture and storage and will preferably be preserved against the contaminating action of microorganisms, such as bacteria and fungi. The carrier can be a solvent or dispersion medium containing, for example, water, ethanol, polyol (e.g., glycerol, propylene glycol, and liquid polyethylene glycol, and the like), and suitable mixtures thereof. The proper fluidity can be maintained, for example, by the use of a coating such as lecithin and/or by the maintenance of the required particle size. In specific embodiments, an agent of the present disclosure may be conjugated to a vehicle for cellular delivery. In these embodiments, the agent may be encapsulated in a suitable vehicle to either aid in the delivery of the agent to target cells, to increase the stability of the agent, or to minimize potential toxicity of the agent. As will be appreciated by a skilled artisan, a variety of vehicles are suitable for delivering an agent of the present disclosure. Non-limiting examples of suitable structured fluid delivery systems may include nanoparticles, liposomes, microemulsions, micelles, dendrimers and other phosphohpid-containing systems. Methods of incorporating agents of the present disclosure into delivery vehicles are known in the art. Although various embodiments are presented below, it will be appreciate that other methods known in the art to incorporate an antigen-binding molecule, as described herein, into a delivery vehicle are contemplated.

Dosage regimens are adjusted to provide the optimum desired response (e.g, a therapeutic response). For example, a single bolus may be administered, several divided doses may be administered over time or the dose may be proportionally reduced or increased as indicated by the exigencies of the therapeutic situation. An antigen-binding molecule of the present disclosure can be administered on multiple occasions. Intervals between single dosages can be daily, weekly, monthly or yearly. Intervals can also be irregular as indicated by measuring blood levels of modified polypeptide or antigen in the patient. Alternatively, the antigen-binding molecule can be administered as a sustained release formulation, in which case less frequent administration is required. Dosage and frequency vary depending on the half-life of the polypeptide in the patient.

It may be advantageous to formulate compositions in dosage unit form for ease of administration and uniformity of dosage. Dosage unit form as used herein refers to physically discrete units suited as unitary' dosages for the subjects to be treated; each unit contains a predetermined quantity of active compound calculated to produce the desired therapeutic effect in association with the required pharmaceutically acceptable carrier. The specification for the dosage unit forms of the invention are dictated by and directly dependent on (a) the unique characteristics of the active compound and the particular therapeutic effect to be achieved, and (b) the limitations inherent in the art of compounding such an active compound for the treatment of sensitivity in individuals.

Dosages and therapeutic regimens of the antigen-binding molecule can be determined by a skilled artisan. In certain embodiments, the antigen-binding molecule is administered by injection (e.g., subcutaneously or intravenously) at a dose of about 0.01 to 40 mg/kg, e.g., 0.01 to 0.1 mg/kg, e.g, about 0.1 to 1 mg/kg, about 1 to 5 mg/kg, about 5 to 25 mg/kg, about 10 to 40 mg/kg, or about 0.4 mg/kg. The dosing schedule can vary from e.g., once a week to once every 2, 3, or 4 weeks. In one embodiment, the antigen-binding molecule is administered at a dose from about 10 to 20 mg/kg every other week. An exemplary, non-limiting range for an effective amount of an antigen-binding molecule of the present disclosure is 0.01-5 mg/kg, more suitably 0.03-2 mg/kg.

It is to be noted that dosage values may vary with the type and severity of the condition to be alleviated. It is to be further understood that for any particular subject, specific dosage regimens should be adjusted over time according to the individual need and the professional judgment of the person administering or supervising the administration of the compositions, and that dosage ranges set forth herein are exemplary only and are not intended to limit the scope or practice of the claimed composition.

The pharmaceutical compositions of the invention may include an effective amount of agent (i.e., the CD20-binding molecule) disclosed herein. The effective amount may be a “therapeutically effective amount” or a “prophylactically effective amount”. A “therapeutically effective amount” refers to an amount effective, at dosages and for periods of time necessary, to achieve the desired therapeutic result. A therapeutically effective amount of the agent may vary according to factors such as the disease state, age, sex, and weight of the individual, and the ability of the agent to elicit a desired response in the individual. A therapeutically effective amount is also one in which any toxic or detrimental effects of the agent is outweighed by the therapeutically beneficial effects. Alternatively, this property of a composition can be evaluated by examining the ability of the compound to inhibit, for example in in vitro by assays known to the skilled practitioner.

By contrast, a “prophylactically effective amount” refers to an amount effective, at dosages and for periods of time necessary, to achieve the desired prophylactic result. Typically, since a prophylactic dose is used in subjects prior to or at an earlier stage of disease, the prophylactically effective amount will be less than the therapeutically effective amount.

The term “treating” as used herein may refer to (1) delaying the appearance of one or more symptoms of the condition; (2) inhibiting the development of the condition or one or more symptoms of the condition; (3) relieving the condition, i.e., causing regression of the condition or at least one or more symptoms of the condition; and/or (4) causing a decrease in the severity of the condition or of one or more symptoms of the condition.

The terms “treating”, “treatment” and the like, are used interchangeably herein to mean relieving, reducing, alleviating, ameliorating or otherwise inhibiting the condition, including one or more symptoms of the condition. The terms “prevent”, “preventing”, “prophylaxis”, “prophylactic”, “preventative” and the like are used interchangeably herein to mean preventing or delaying the onset of the condition, or the risk of developing the condition.

The terms “treating”, “treatment” and the like also include relieving, reducing, alleviating, ameliorating or otherwise inhibiting the effects of the condition for at least a period of time. It is also to be understood that terms “treating”, “treatment” and the like do not imply that the condition, or a symptom thereof, is permanently relieved, reduced, alleviated, ameliorated or otherwise inhibited and therefore also encompasses the temporary relief, reduction, alleviation, amelioration or otherwise inhibition of the condition, or of a symptom thereof.

The terms “subject”, “patient”, ‘host” or “individual” used interchangeably herein, refer to any subject, particularly a vertebrate subject, and even more particularly a mammalian subject, for whom therapy or prophylaxis is desired. In one embodiment, the subject is a human subject.

The following numbered embodiments are contemplated and are non-limiting:
1. An antigen-binding molecule that specifically binds to CD20, wherein the antigen-binding molecule comprises an immunoglobulin heavy chain variable domain (VH) and an immunoglobulin light chain variable domain (VL), wherein the VH comprises a complementarity determining region 1 (VH CDR1) comprising the amino acid sequence of SEQ ID NO: 9, a VH CDR2 comprising the amino acid sequence of SEQ ID NO: 13 and a VH CDR3 comprising the amino acid sequence of SEQ ID NO: 17; and wherein the VL comprises a complementarity determining region 1 (VL CDR1) comprising the amino acid sequence of SEQ ID NO: 21, a VL CDR2 comprising the amino acid sequence of SEQ ID NO: 25, and a VL CDR3 comprising the amino acid sequence of SEQ ID NO: 29:

VH CDR1
(SEQ ID NO: 9)
SSYAM
VH CDR2
(SEQ ID NO: 13)
EITNYAAGSGTWYGAAVK
VH CDR3
(SEQ ID NO: 17)
KGPISGSIGYLSSIDA
VL CDR1
(SEQ ID NO: 21)
SGGSSRYGYG
VL CDR2
(SEQ ID NO: 25)
WNDKRPS
VL CDR3
(SEQ ID NO: 29)
GNYDNNDPA.

2. The antigen-binding molecule of clause 1, any other suitable clause, or any combination of suitable clauses, wherein the VH comprises an amino acid sequence having at least 80% sequence identity to SEQ ID NO: 1.
3. The antigen-binding molecule of clause 1, any other suitable clause, or any combination of suitable clauses, wherein the VH comprises an amino acid sequence having at least 85% sequence identity to SEQ ID NO: 1.
4. The antigen-binding molecule of clause 1, any other suitable clause, or any combination of suitable clauses, wherein the VH comprises an amino acid sequence having at least 90% sequence identity to SEQ ID NO: 1.
5. The antigen-binding molecule of clause 1, any other suitable clause, or any combination of suitable clauses, wherein the VH comprises an amino acid sequence having at least 91% sequence identity to SEQ ID NO: 1.
6. The antigen-binding molecule of clause 1, any other suitable clause, or any combination of suitable clauses, wherein the VH comprises an amino acid sequence having at least 92% sequence identity to SEQ ID NO: 1.
7. The antigen-binding molecule of clause 1, any other suitable clause, or any combination of suitable clauses, wherein the VH comprises an amino acid sequence having at least 93% sequence identity to SEQ ID NO: 1.
8. The antigen-binding molecule of clause 1, any other suitable clause, or any combination of suitable clauses, wherein the VH comprises an amino acid sequence having at least 94% sequence identity to SEQ ID NO: 1.
9. The antigen-binding molecule of clause 1, any other suitable clause, or any combination of suitable clauses, wherein the VH comprises an amino acid sequence having at least 95% sequence identity to SEQ ID NO: 1.
10. The antigen-binding molecule of clause 1, any other suitable clause, or any combination of suitable clauses, wherein the VH comprises an amino acid sequence having at least 96% sequence identity to SEQ ID NO: 1.
11. The antigen-binding molecule of clause 1, any other suitable clause, or any combination of suitable clauses, wherein the VH comprises an amino acid sequence having at least 97% sequence identity to SEQ ID NO: 1.
12. The antigen-binding molecule of clause 1, any other suitable clause, or any combination of suitable clauses, wherein the VH comprises an amino acid sequence having at least 98% sequence identity to SEQ ID NO: 1.
13. The antigen-binding molecule of clause 1, any other suitable clause, or any combination of suitable clauses, wherein the VH comprises an amino acid sequence having at least 99% sequence identity to SEQ ID NO: 1.
14. The antigen-binding molecule of clause 1, any other suitable clause, or any combination of suitable clauses, wherein the VL comprises an amino acid sequence having at least 80% sequence identity to SEQ ID NO: 5.
15. The antigen-binding molecule of clause 1, any other suitable clause, or any combination of suitable clauses, wherein the VL comprises an amino acid sequence having at least 85% sequence identity to SEQ ID NO: 5.
16. The antigen-binding molecule of clause 1, any other suitable clause, or any combination of suitable clauses, wherein the VL comprises an amino acid sequence having at least 90% sequence identity to SEQ ID NO: 5.
17. The antigen-binding molecule of clause 1, any other suitable clause, or any combination of suitable clauses, wherein the VL comprises an amino acid sequence having at least 91% sequence identity to SEQ ID NO: 5.
18. The antigen-binding molecule of clause 1, any other suitable clause, or any combination of suitable clauses, wherein the VL comprises an amino acid sequence having at least 92% sequence identity to SEQ ID NO: 5.
19. The antigen-binding molecule of clause 1, any other suitable clause, or any combination of suitable clauses, wherein the VL comprises an amino acid sequence having at least 93% sequence identity to SEQ ID NO: 5.
20. The antigen-binding molecule of clause 1, any other suitable clause, or any combination of suitable clauses, wherein the VL comprises an amino acid sequence having at least 94% sequence identity to SEQ ID NO: 5.
21. The antigen-binding molecule of clause 1, any other suitable clause, or any combination of suitable clauses, wherein the VL comprises an amino acid sequence having at least 95% sequence identity to SEQ ID NO: 5.
22. The antigen-binding molecule of clause 1, any other suitable clause, or any combination of suitable clauses, wherein the VL comprises an amino acid sequence having at least 96% sequence identity to SEQ ID NO: 5.
23. The antigen-binding molecule of clause 1, any other suitable clause, or any combination of suitable clauses, wherein the VL comprises an amino acid sequence having at least 97% sequence identity to SEQ ID NO: 5.
24. The antigen-binding molecule of clause 1, any other suitable clause, or any combination of suitable clauses, wherein the VL comprises an amino acid sequence having at least 98% sequence identity to SEQ ID NO: 5.
25. The antigen-binding molecule of clause 1, any other suitable clause, or any combination of suitable clauses, wherein the VL comprises an amino acid sequence having at least 99% sequence identity to SEQ ID NO: 5.
26. An antigen-binding molecule that specifically binds to CD20, wherein the antigen-binding molecule comprises an immunoglobulin heavy chain variable domain (VH) and an immunoglobulin light chain variable domain (VL), wherein the VH comprises a complementarity determining region 1 (VH CDR1) comprising the amino acid sequence of SEQ ID NO: 10, a VH CDR2 comprising the amino acid sequence of SEQ ID NO: 14 and a VH CDR3 comprising the amino acid sequence of SEQ ID NO: 18; and wherein the VL comprises a complementarity determining region 1 (VL CDR1) comprising the amino acid sequence of SEQ ID NO: 22, a VL CDR2 comprising the amino acid sequence of SEQ ID NO: 26, and a VL CDR3 comprising the amino acid sequence of SEQ ID NO: 30:

VH CDR1
(SEQ ID NO: 10)
SSSYIN
VH CDR2
(SEQ ID NO: 14)
QINKDGGGSWYAPVVK
VH CDR3
(SEQ ID NO: 18)
KNADSGCVGVGGCIDT
VL CDR1
(SEQ ID NO: 22)
SGGSYYYGGNYYYG
VL CDR2
(SEQ ID NO: 26)
NNNKRPS
VL CDR3
(SEQ ID NO: 30)
GGYDSSYVAI.

27. The antigen-binding molecule of clause 26, any other suitable clause, or any combination of suitable clauses, wherein the VH comprises an amino acid sequence having at least 80% sequence identity to SEQ ID NO: 2.
28. The antigen-binding molecule of clause 26, any other suitable clause, or any combination of suitable clauses, wherein the VH comprises an amino acid sequence having at least 85% sequence identity to SEQ ID NO: 2.
29. The antigen-binding molecule of clause 26, any other suitable clause, or any combination of suitable clauses, wherein the VH comprises an amino acid sequence having at least 90% sequence identity to SEQ ID NO: 2.
30. The antigen-binding molecule of clause 26, any other suitable clause, or any combination of suitable clauses, wherein the VH comprises an amino acid sequence having at least 91% sequence identity to SEQ ID NO: 2.
31. The antigen-binding molecule of clause 26, any other suitable clause, or any combination of suitable clauses, wherein the VH comprises an amino acid sequence having at least 92% sequence identity to SEQ ID NO: 2.
32. The antigen-binding molecule of clause 26, any other suitable clause, or any combination of suitable clauses, wherein the VH comprises an amino acid sequence having at least 93% sequence identity to SEQ ID NO: 2.
33. The antigen-binding molecule of clause 26, any other suitable clause, or any combination of suitable clauses, wherein the VH comprises an amino acid sequence having at least 94% sequence identity to SEQ ID NO: 2.
34. The antigen-binding molecule of clause 26, any other suitable clause, or any combination of suitable clauses, wherein the VH comprises an amino acid sequence having at least 95% sequence identity to SEQ ID NO: 2.
35. The antigen-binding molecule of clause 26, any other suitable clause, or any combination of suitable clauses, wherein the VH comprises an amino acid sequence having at least 96% sequence identity to SEQ ID NO: 2.
36. The antigen-binding molecule of clause 26, any other suitable clause, or any combination of suitable clauses, wherein the VH comprises an amino acid sequence having at least 97% sequence identity to SEQ ID NO: 2.
37. The antigen-binding molecule of clause 26, any other suitable clause, or any combination of suitable clauses, wherein the VH comprises an amino acid sequence having at least 98% sequence identity to SEQ ID NO: 2.
38. The antigen-binding molecule of clause 26, any other suitable clause, or any combination of suitable clauses, wherein the VH comprises an amino acid sequence having at least 99% sequence identity to SEQ ID NO: 2.
39. The antigen-binding molecule of clause 26, any other suitable clause, or any combination of suitable clauses, wherein the VL comprises an amino acid sequence having at least 80% sequence identity to SEQ ID NO: 6.
40. The antigen-binding molecule of clause 26, any other suitable clause, or any combination of suitable clauses, wherein the VL comprises an amino acid sequence having at least 85% sequence identity to SEQ ID NO: 6.
41. The antigen-binding molecule of clause 26, any other suitable clause, or any combination of suitable clauses, wherein the VL comprises an amino acid sequence having at least 90% sequence identity to SEQ ID NO: 6.
42. The antigen-binding molecule of clause 26, any other suitable clause, or any combination of suitable clauses, wherein the VL comprises an amino acid sequence having at least 91% sequence identity to SEQ ID NO: 6.
43. The antigen-binding molecule of clause 26, any other suitable clause, or any combination of suitable clauses, wherein the VL comprises an amino acid sequence having at least 92% sequence identity to SEQ ID NO: 6.
44. The antigen-binding molecule of clause 26, any other suitable clause, or any combination of suitable clauses, wherein the VL comprises an amino acid sequence having at least 93% sequence identity to SEQ ID NO: 6.
45. The antigen-binding molecule of clause 26, any other suitable clause, or any combination of suitable clauses, wherein the VL comprises an amino acid sequence having at least 94% sequence identity to SEQ ID NO: 6.
46. The antigen-binding molecule of clause 26, any other suitable clause, or any combination of suitable clauses, wherein the VL comprises an amino acid sequence having at least 95% sequence identity to SEQ ID NO: 6.
47. The antigen-binding molecule of clause 26, any other suitable clause, or any combination of suitable clauses, wherein the VL comprises an amino acid sequence having at least 96% sequence identity to SEQ ID NO: 6.
48. The antigen-binding molecule of clause 26, any other suitable clause, or any combination of suitable clauses, wherein the VL comprises an amino acid sequence having at least 97% sequence identity to SEQ ID NO: 6.
49. The antigen-binding molecule of clause 26, any other suitable clause, or any combination of suitable clauses, wherein the VL comprises an amino acid sequence having at least 98% sequence identity to SEQ ID NO: 6.
50. The antigen-binding molecule of clause 26, any other suitable clause, or any combination of suitable clauses, wherein the VL comprises an amino acid sequence having at least 99% sequence identity to SEQ ID NO: 6.
51. The antigen-binding molecule of clause 26, any other suitable clause, or any combination of suitable clauses, wherein the VH comprises an amino acid sequence having at least 80% sequence identity to SEQ ID NO: 33.
52. The antigen-binding molecule of clause 26, any other suitable clause, or any combination of suitable clauses, wherein the VH comprises an amino acid sequence having at least 85% sequence identity to SEQ ID NO: 33.
53. The antigen-binding molecule of clause 26, any other suitable clause, or any combination of suitable clauses, wherein the VH comprises an amino acid sequence having at least 90% sequence identity to SEQ ID NO: 33.
54. The antigen-binding molecule of clause 26, any other suitable clause, or any combination of suitable clauses, wherein the VH comprises an amino acid sequence having at least 91% sequence identity to SEQ ID NO: 33.
55. The antigen-binding molecule of clause 26, any other suitable clause, or any combination of suitable clauses, wherein the VH comprises an amino acid sequence having at least 92% sequence identity to SEQ ID NO: 33.
56. The antigen-binding molecule of clause 26, any other suitable clause, or any combination of suitable clauses, wherein the VH comprises an amino acid sequence having at least 93% sequence identity to SEQ ID NO: 33.
57. The antigen-binding molecule of clause 26, any other suitable clause, or any combination of suitable clauses, wherein the VH comprises an amino acid sequence having at least 94% sequence identity to SEQ ID NO: 33.
58. The antigen-binding molecule of clause 26, any other suitable clause, or any combination of suitable clauses, wherein the VH comprises an amino acid sequence having at least 95% sequence identity to SEQ ID NO: 33.
59. The antigen-binding molecule of clause 26, any other suitable clause, or any combination of suitable clauses, wherein the VH comprises an amino acid sequence having at least 96% sequence identity to SEQ ID NO: 33.
60. The antigen-binding molecule of clause 26, any other suitable clause, or any combination of suitable clauses, wherein the VH comprises an amino acid sequence having at least 97% sequence identity to SEQ ID NO: 33.
61. The antigen-binding molecule of clause 26, any other suitable clause, or any combination of suitable clauses, wherein the VH comprises an amino acid sequence having at least 98% sequence identity to SEQ ID NO: 33.
62. The antigen-binding molecule of clause 26, any other suitable clause, or any combination of suitable clauses, wherein the VH comprises an amino acid sequence having at least 99% sequence identity to SEQ ID NO: 33.
63. The antigen-binding molecule of clause 26, any other suitable clause, or any combination of suitable clauses, wherein the VL comprises an amino acid sequence having at least 80% sequence identity to SEQ ID NO: 34.
64. The antigen-binding molecule of clause 26, any other suitable clause, or any combination of suitable clauses, wherein the VL comprises an amino acid sequence having at least 85% sequence identity to SEQ ID NO: 34.
65. The antigen-binding molecule of clause 26, any other suitable clause, or any combination of suitable clauses, wherein the VL comprises an amino acid sequence having at least 90% sequence identity to SEQ ID NO: 34.
66. The antigen-binding molecule of clause 26, any other suitable clause, or any combination of suitable clauses, wherein the VL comprises an amino acid sequence having at least 91% sequence identity to SEQ ID NO: 34.
67. The antigen-binding molecule of clause 26, any other suitable clause, or any combination of suitable clauses, wherein the VL comprises an amino acid sequence having at least 92% sequence identity to SEQ ID NO: 34.
68. The antigen-binding molecule of clause 26, any other suitable clause, or any combination of suitable clauses, wherein the VL comprises an amino acid sequence having at least 93% sequence identity to SEQ ID NO: 34.
69. The antigen-binding molecule of clause 26, any other suitable clause, or any combination of suitable clauses, wherein the VL comprises an amino acid sequence having at least 94% sequence identity to SEQ ID NO: 34.
70. The antigen-binding molecule of clause 26, any other suitable clause, or any combination of suitable clauses, wherein the VL comprises an amino acid sequence having at least 95% sequence identity to SEQ ID NO: 34.
71. The antigen-binding molecule of clause 26, any other suitable clause, or any combination of suitable clauses, wherein the VL comprises an amino acid sequence having at least 96% sequence identity to SEQ ID NO: 34.
72. The antigen-binding molecule of clause 26, any other suitable clause, or any combination of suitable clauses, wherein the VL comprises an amino acid sequence having at least 97% sequence identity to SEQ ID NO: 34.
73. The antigen-binding molecule of clause 26, any other suitable clause, or any combination of suitable clauses, wherein the VL comprises an amino acid sequence having at least 98% sequence identity to SEQ ID NO: 34.
74. The antigen-binding molecule of clause 26, any other suitable clause, or any combination of suitable clauses, wherein the VL comprises an amino acid sequence having at least 99% sequence identity to SEQ ID NO: 34.
75. An antigen-binding molecule that specifically binds to CD20, wherein the antigen-binding molecule comprises an immunoglobulin heavy chain variable domain (VH) and an immunoglobulin light chain variable domain (VL), wherein the VH comprises a complementarity determining region 1 (VH CDR1) comprising the amino acid sequence of SEQ ID NO: 11, a VH CDR2 comprising the amino acid sequence of SEQ ID NO: 15 and a VH CDR3 comprising the amino acid sequence of SEQ ID NO: 19; and wherein the VL comprises a complementarity determining region 1 (VL CDR1) comprising the amino acid sequence of SEQ ID NO: 23, a VL CDR2 comprising the amino acid sequence of SEQ ID NO: 27, and a VL CDR3 comprising the amino acid sequence of SEQ ID NO: 31:

VH CDR1
(SEQ ID NO: 11)
NSNGMG
VH CDR2
(SEQ ID NO: 15)
GIHSSGRYTYYGTAVK
VH CDR3
(SEQ ID NO: 19)
KNADSAYGYWYAGSIDA
VL CDR1
(SEQ ID NO: 23)
SGGYSSYGYS
VL CDR2
(SEQ ID NO: 27)
NNNNRPS
VL CDR3
(SEQ ID NO: 31)
AFTDYSSLAGV.

76. The antigen-binding molecule of clause 75, any other suitable clause, or any combination of suitable clauses, wherein the VH comprises an amino acid sequence having at least 80% sequence identity to SEQ ID NO: 3.
77. The antigen-binding molecule of clause 75, any other suitable clause, or any combination of suitable clauses, wherein the VH comprises an amino acid sequence having at least 85% sequence identity to SEQ ID NO: 3.
78. The antigen-binding molecule of clause 75, any other suitable clause, or any combination of suitable clauses, wherein the VH comprises an amino acid sequence having at least 90% sequence identity to SEQ ID NO: 3.
79. The antigen-binding molecule of clause 75, any other suitable clause, or any combination of suitable clauses, wherein the VH comprises an amino acid sequence having at least 91% sequence identity to SEQ ID NO: 3.
80. The antigen-binding molecule of clause 75, any other suitable clause, or any combination of suitable clauses, wherein the VH comprises an amino acid sequence having at least 92% sequence identity to SEQ ID NO: 3.
81. The antigen-binding molecule of clause 75, any other suitable clause, or any combination of suitable clauses, wherein the VH comprises an amino acid sequence having at least 93% sequence identity to SEQ ID NO: 3.
82. The antigen-binding molecule of clause 75, any other suitable clause, or any combination of suitable clauses, wherein the VH comprises an amino acid sequence having at least 94% sequence identity to SEQ ID NO: 3.
83. The antigen-binding molecule of clause 75, any other suitable clause, or any combination of suitable clauses, wherein the VH comprises an amino acid sequence having at least 95% sequence identity to SEQ ID NO: 3.
84. The antigen-binding molecule of clause 75, any other suitable clause, or any combination of suitable clauses, wherein the VH comprises an amino acid sequence having at least 96% sequence identity to SEQ ID NO: 3.
85. The antigen-binding molecule of clause 75, any other suitable clause, or any combination of suitable clauses, wherein the VH comprises an amino acid sequence having at least 97% sequence identity to SEQ ID NO: 3.
86. The antigen-binding molecule of clause 75, any other suitable clause, or any combination of suitable clauses, wherein the VH comprises an amino acid sequence having at least 98% sequence identity to SEQ ID NO: 3.
87. The antigen-binding molecule of clause 75, any other suitable clause, or any combination of suitable clauses, wherein the VH comprises an amino acid sequence having at least 99% sequence identity to SEQ ID NO: 3.
88. The antigen-binding molecule of clause 75, any other suitable clause, or any combination of suitable clauses, wherein the VL comprises an amino acid sequence having at least 80% sequence identity to SEQ ID NO: 7.
89. The antigen-binding molecule of clause 75, any other suitable clause, or any combination of suitable clauses, wherein the VL comprises an amino acid sequence having at least 85% sequence identity to SEQ ID NO: 7.
90. The antigen-binding molecule of clause 75, any other suitable clause, or any combination of suitable clauses, wherein the VL comprises an amino acid sequence having at least 90% sequence identity to SEQ ID NO: 7.
91. The antigen-binding molecule of clause 75, any other suitable clause, or any combination of suitable clauses, wherein the VL comprises an amino acid sequence having at least 91% sequence identity to SEQ ID NO: 7.
92. The antigen-binding molecule of clause 75, any other suitable clause, or any combination of suitable clauses, wherein the VL comprises an amino acid sequence having at least 92% sequence identity to SEQ ID NO: 7.
93. The antigen-binding molecule of clause 75, any other suitable clause, or any combination of suitable clauses, wherein the VL comprises an amino acid sequence having at least 93% sequence identity to SEQ ID NO: 7.
94. The antigen-binding molecule of clause 75, any other suitable clause, or any combination of suitable clauses, wherein the VL comprises an amino acid sequence having at least 94% sequence identity to SEQ ID NO: 7.
95. The antigen-binding molecule of clause 75, any other suitable clause, or any combination of suitable clauses, wherein the VL comprises an amino acid sequence having at least 95% sequence identity to SEQ ID NO: 7.
96. The antigen-binding molecule of clause 75, any other suitable clause, or any combination of suitable clauses, wherein the VL comprises an amino acid sequence having at least 96% sequence identity to SEQ ID NO: 7.
97. The antigen-binding molecule of clause 75, any other suitable clause, or any combination of suitable clauses, wherein the VL comprises an amino acid sequence having at least 97% sequence identity to SEQ ID NO: 7.
98. The antigen-binding molecule of clause 75, any other suitable clause, or any combination of suitable clauses, wherein the VL comprises an amino acid sequence having at least 98% sequence identity to SEQ ID NO: 7.
99. The antigen-binding molecule of clause 75, any other suitable clause, or any combination of suitable clauses, wherein the VL comprises an amino acid sequence having at least 99% sequence identity to SEQ ID NO: 7.
100. An antigen-binding molecule that specifically binds to CD20, wherein the antigen-binding molecule comprises an immunoglobulin heavy chain variable domain (VH) and an immunoglobulin light chain variable domain (VL), wherein the VH comprises a complementarity determining region 1 (VH CDR1) comprising the amino acid sequence of SEQ ID NO: 12, a VH CDR2 comprising the amino acid sequence of SEQ ID NO: 16 and a VH CDR3 comprising the amino acid sequence of SEQ ID NO: 20; and wherein the VL comprises a complementarity determining region 1 (VL CDR1) comprising the amino acid sequence of SEQ ID NO: 24, a VL CDR2 comprising the amino acid sequence of SEQ ID NO: 28, and a VL CDR3 comprising the amino acid sequence of SEQ ID NO: 32:

VH CDR1
(SEQ ID NO: 12)
DRHGMF
VH CDR2
(SEQ ID NO: 16)
GIRSTGSYPKYGPAVK
VH CDR3
(SEQ ID NO: 20)
RNVYIGDYDGDSIDA
VL CDR1
(SEQ ID NO: 24)
SGSSSGYGSYYG
VL CDR2
(SEQ ID NO: 28)
ENTNRPS
VL CDR3
(SEQ ID NO: 32)
GSADDSGNSAI.

101. The antigen-binding molecule of clause 100, any other suitable clause, or any combination of suitable clauses, wherein the VH comprises an amino acid sequence having at least 80% sequence identity to SEQ ID NO: 4.
102. The antigen-binding molecule of clause 100, any other suitable clause, or any combination of suitable clauses, wherein the VH comprises an amino acid sequence having at least 85% sequence identity to SEQ ID NO: 4.
103. The antigen-binding molecule of clause 100, any other suitable clause, or any combination of suitable clauses, wherein the VH comprises an amino acid sequence having at least 90% sequence identity to SEQ ID NO: 4.
104. The antigen-binding molecule of clause 100, any other suitable clause, or any combination of suitable clauses, wherein the VH comprises an amino acid sequence having at least 91% sequence identity to SEQ ID NO: 4.
105. The antigen-binding molecule of clause 100, any other suitable clause, or any combination of suitable clauses, wherein the VH comprises an amino acid sequence having at least 92% sequence identity to SEQ ID NO: 4.
106. The antigen-binding molecule of clause 100, any other suitable clause, or any combination of suitable clauses, wherein the VH comprises an amino acid sequence having at least 93% sequence identity to SEQ ID NO: 4.
107. The antigen-binding molecule of clause 100, any other suitable clause, or any combination of suitable clauses, wherein the VH comprises an amino acid sequence having at least 94% sequence identity to SEQ ID NO: 4.
108. The antigen-binding molecule of clause 100, any other suitable clause, or any combination of suitable clauses, wherein the VH comprises an amino acid sequence having at least 95% sequence identity to SEQ ID NO: 4.
109. The antigen-binding molecule of clause 100, any other suitable clause, or any combination of suitable clauses, wherein the VH comprises an amino acid sequence having at least 96% sequence identity to SEQ ID NO: 4.
110. The antigen-binding molecule of clause 100, any other suitable clause, or any combination of suitable clauses, wherein the VH comprises an amino acid sequence having at least 97% sequence identity to SEQ ID NO: 4.
111. The antigen-binding molecule of clause 100, any other suitable clause, or any combination of suitable clauses, wherein the VH comprises an amino acid sequence having at least 98% sequence identity to SEQ ID NO: 4.
112. The antigen-binding molecule of clause 100, any other suitable clause, or any combination of suitable clauses, wherein the VH comprises an amino acid sequence having at least 99% sequence identity to SEQ ID NO: 4.
113. The antigen-binding molecule of clause 100, any other suitable clause, or any combination of suitable clauses, wherein the VL comprises an amino acid sequence having at least 80% sequence identity to SEQ ID NO: 8.
114. The antigen-binding molecule of clause 100, any other suitable clause, or any combination of suitable clauses, wherein the VL comprises an amino acid sequence having at least 85% sequence identity to SEQ ID NO: 8.
115. The antigen-binding molecule of clause 100, any other suitable clause, or any combination of suitable clauses, wherein the VL comprises an amino acid sequence having at least 90% sequence identity to SEQ ID NO: 8.
116. The antigen-binding molecule of clause 100, any other suitable clause, or any combination of suitable clauses, wherein the VL comprises an amino acid sequence having at least 91% sequence identity to SEQ ID NO: 8.
117. The antigen-binding molecule of clause 100, any other suitable clause, or any combination of suitable clauses, wherein the VL comprises an amino acid sequence having at least 92% sequence identity to SEQ ID NO: 8.
118. The antigen-binding molecule of clause 100, any other suitable clause, or any combination of suitable clauses, wherein the VL comprises an amino acid sequence having at least 93% sequence identity to SEQ ID NO: 8.
119. The antigen-binding molecule of clause 100, any other suitable clause, or any combination of suitable clauses, wherein the VL comprises an amino acid sequence having at least 94% sequence identity to SEQ ID NO: 8.
120. The antigen-binding molecule of clause 100, any other suitable clause, or any combination of suitable clauses, wherein the VL comprises an amino acid sequence having at least 95% sequence identity to SEQ ID NO: 8.
121. The antigen-binding molecule of clause 100, any other suitable clause, or any combination of suitable clauses, wherein the VL comprises an amino acid sequence having at least 96% sequence identity to SEQ ID NO: 8.
122. The antigen-binding molecule of clause 100, any other suitable clause, or any combination of suitable clauses, wherein the VL comprises an amino acid sequence having at least 97% sequence identity to SEQ ID NO: 8.
123. The antigen-binding molecule of clause 100, any other suitable clause, or any combination of suitable clauses, wherein the VL comprises an amino acid sequence having at least 98% sequence identity to SEQ ID NO: 8.
124. The antigen-binding molecule of clause 100, any other suitable clause, or any combination of suitable clauses, wherein the VL comprises an amino acid sequence having at least 99% sequence identity to SEQ ID NO: 8.
125. The antigen-binding molecule of any one of clauses 1 to 124, any other suitable clause, or any combination of suitable clauses, wherein the antigen-binding molecule is an antibody or a CD20-binding fragment thereof.
126. The antigen-binding molecule of clause 125, any other suitable clause, or any combination of suitable clauses, wherein the CD20-binding fragment is selected from the group consisting of an Fab fragment, an scFab, an Fab′, a single chain variable fragment (scFv) and a one-armed antibody.
127. The antigen-binding molecule of any one of clauses 1 to 124, any other suitable clause, or any combination of suitable clauses, wherein the antigen-binding molecule is a humanized antibody or CD20-binding fragment thereof.
128. An isolated nucleic acid molecule comprising a nucleic acid sequence encoding the antigen-binding molecule of any one of clauses 1 to 127.
129. An expression construct comprising a nucleic acid sequence encoding the antigen-binding molecule of any one of clauses 1 to 127, operably linked to one or more regulatory sequences.
130. A host cell comprising the expression construct of clause 129.
131. A vector comprising a nucleic acid sequence encoding the antigen-binding molecule of any one of clauses 1 to 127.
132. A pharmaceutical composition comprising the antigen-binding molecule of any one of clauses 1 to 127, and a pharmaceutically acceptable carrier.
133. A method of treating a disease in a patient, the method comprising administering to the patient in need thereof the antigen-binding molecule of any one of clauses 1 to 127, or a CD20-binding fragment thereof, the vector of clause 131, or the pharmaceutical composition of clause 132.
134. The method of clause 133, any other suitable clause, or any combination of suitable clauses, wherein the disease is a cancer.
135. The method of clause 134, any other suitable clause, or any combination of suitable clauses, wherein the cancer is a CD20 positive B-cell cancer.
136. The method of clause 133, any other suitable clause, or any combination of suitable clauses, wherein the disease is a blood cancer.
137. The method of clause 136, any other suitable clause, or any combination of suitable clauses, wherein the blood cancer is a CD20 positive B-cell blood cancer.
138. The method of clause 133, any other suitable clause, or any combination of suitable clauses, wherein the disease is Non-Hodgkin's Lymphoma (NHL).
139. The method of clause 133, any other suitable clause, or any combination of suitable clauses, wherein the disease is Hodgkin's Lymphoma (HL).
140. The method of clause 133, any other suitable clause, or any combination of suitable clauses, wherein the disease is CD20 positive B-cell NHL.
141. The method of clause 133, any other suitable clause, or any combination of suitable clauses, wherein the disease is Chronic Lymphocytic Leukemia (CLL).
142. The method of clause 133, any other suitable clause, or any combination of suitable clauses, wherein the disease is Rheumatoid Arthritis.
143. The method of clause 133, any other suitable clause, or any combination of suitable clauses, wherein the disease is Microscopic Polyangiitis (MP).
144. The method of clause 133, any other suitable clause, or any combination of suitable clauses, wherein the disease is Granulomatosis with Polyangiitis (GPA) 145. The method of clause 133, any other suitable clause, or any combination of suitable clauses, wherein the disease is Pemphigus Vulgaris (PV).
146. A method of treating or preventing a cancer induced to proliferate by CD20 and conditions associated therewith, the method comprising administering to a subject in need thereof the antigen-binding molecule of any one of clauses 1 to 127, or a CD20-binding fragment thereof, the vector of clause 131, or the pharmaceutical composition of clause 132.
147. The method of clause 146, any other suitable clause, or any combination of suitable clauses, wherein the cancer is a CD20 positive B-cell cancer.
148. The method of clause 146, any other suitable clause, or any combination of suitable clauses, wherein the cancer is a blood cancer.
149. The method of clause 148, any other suitable clause, or any combination of suitable clauses, wherein the blood cancer is a CD20 positive B-cell blood cancer.
150. The method of clause 146, any other suitable clause, or any combination of suitable clauses, wherein the cancer is a lymphoma.
151. The method of clause 146, any other suitable clause, or any combination of suitable clauses, wherein the lymphoma is a CD20 positive B-cell lymphoma.
152. A kit comprising the antigen-binding molecule of any one of clauses 1 to 127, or a CD20-binding fragment thereof, or the vector of clause 131, or the pharmaceutical composition of clause 132.

The use of the term “or” in the claims or clauses herein is used to mean “and/or” unless explicitly indicated to refer to alternatives only or the alternatives are mutually exclusive, although the disclosure supports a definition that refers to only alternatives and “and/or.”

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

### Example 1

### Exemplary Experimental Procedures

The instant example provides exemplary materials and methods utilized in Examples 2-8 as described herein.

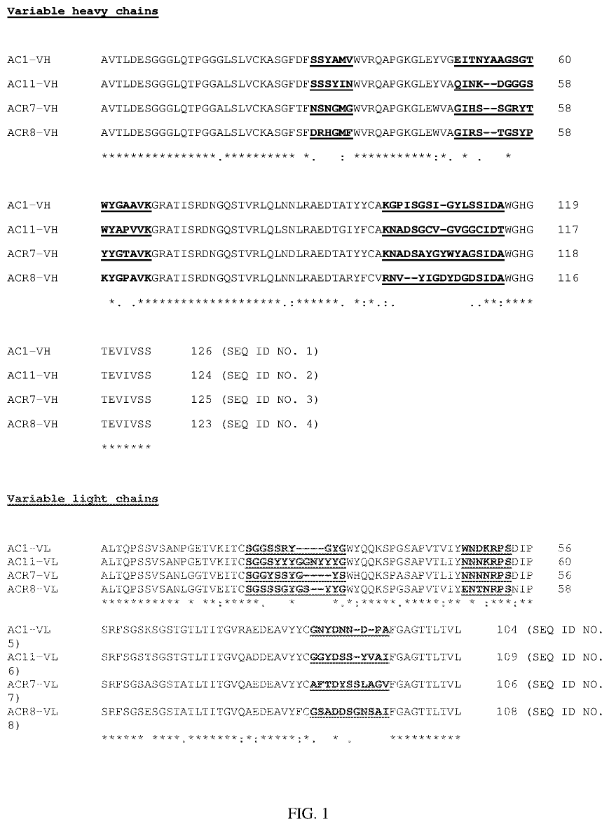

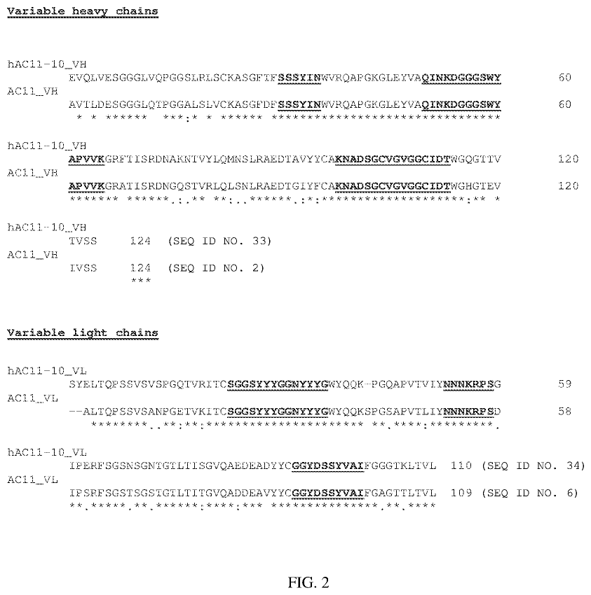

Materials and methods: Reagents, kits, cells, Abs and vectors used in this work are described in Supplementary Methods. The protein sequences of the chicken-derived anti-hCD20 mAbs are provided in FIGS. 1 and 2. Unless otherwise specified, all oligonucleotides were purchased from Integrated DNA Technologies, enzymes and reaction buffers from New England Biolabs, and chemicals from Sigma-Aldrich. Agarose gel extractions were performed with the Zymoclean Gel DNA Recovery Kit (Zymo Research) and cleanup of PCR reactions were performed with the QIAquick PCR Purification Kit (Qiagen). HEK293T cells were cultured from a lab stock in DMEM medium supplemented with 10% heat-inactivated fetal bovine serum (HI-FBS). PE-labeled anti-CD19 Ab was purchased from BD Biosciences. Unconjugated anti-CD3 Ab (BD Biosciences) was labeled with Alexa Fluor 647 (AF647) NHS ester and unconjugated goat anti-human IgG Feg (Jackson ImmunoResearch) was labeled with Atto 488 (A488) NHS ester. Rat anti-mouse CD20 clone AISB12 (eBioscience) was purchased from Thermo Fisher. Expi293F cells were from Thermo Fisher and cultured in Expi293 Expression Medium according to the manufacturer's recommendations. Raji cells were from American Type Culture Collection (ATCC) and cultured in RPMI 1640 (Sigma-Aldrich) supplemented with 10% HI-FBS.

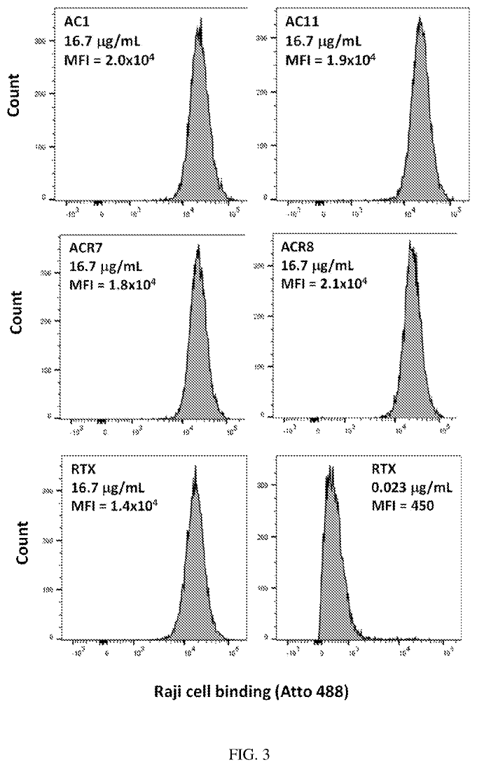

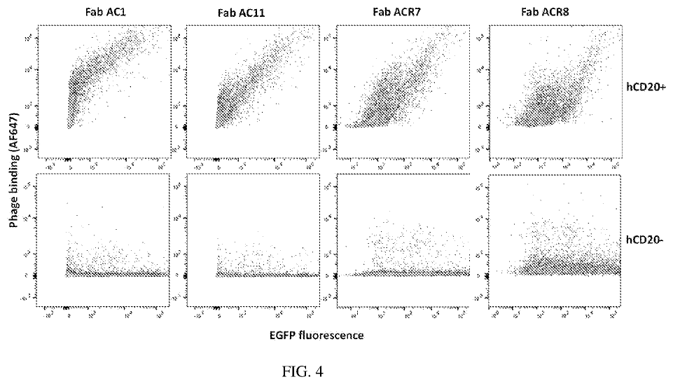

Binding of anti-hCD20 mAbs to Raji cells: Anti-hCD20 Abs diluted in DPBS/1% bovine serum albumin (BSA) were incubated with 6×10⁵ Raji cells in V-bottom 96-well plates at 4° C. for 1 hour in a final volume of 50 mL. The cells were washed with cold DPBS/1% BSA and incubated at 4° C. for 1 hour with 50 mg/mL goat anti-human Fcg (Jackson ImmunoResearch) labeled with Atto 488 (A488) NHS ester. Binding of mAbs to live single cells gated by light scatter characteristics was measured using a BD LSRFortessa X-20 flow cytometer. Representative flow cytometry histograms are presented in FIG. 3. The binding of bacteriophage displaying the Fab portions of the chicken Abs to hCD20-transfected Chinese hamster ovary (CHO) cells was also measured (FIG. 4).

ADCC bioassays: The PBMC ADCC Bioassay containing Raji (HaloTag-HiBiT) and PBMC, ADCC qualified cells was purchased from Promega (#CS3055A14) and used per the manufacturer's recommended protocol. Briefly, frozen Raji cells were thawed and incubated in RPMI 1640/10% heat-inactivated (HI)-FBS and thawed PBMC were incubated in the same medium supplemented with 55 mM b-mercaptoethanol (b-ME) and 5 ng/mL IL-2 (Sigma-Aldrich) at 37° C./5% CO₂ overnight. Cells were then washed with assay buffer (RPMI/10% HI-FBS+55 mM b-ME+5 ng/mL IL-2) and incubated at 25:1 E:T ratio with Abs or 100 mg/mL digitonin (maximum lysis treatment control) in white round-bottom 96-well plates at 37° C./5% CO₂ for 5 hours. The HiBiT fusion protein in the supernatant was quantified via luminescence measurement following addition of Nano-Glo® HiBiT Extracellular Substrate and LgBiT Protein. The ADCC Reporter Bioassay, Complete Kit (Raji) (Promega, G7015) containing thaw-and-use Raji (target) cells and FcgRIIIa (V158 variant)-displaying nuclear factor of activated T-cells (NFAT)-responsive luciferase reporter (effector) cells was used per the manufacturer's recommendations. Antibody dose responses were setup in duplicate. Luminescence readings were taken with a Berthold Centro LB 960 luminometer using a 0.5 s integration time.

Complement-dependent cytotoxicity assay: Raji cells were first harvested and resuspended in assay medium composed of RPMI 1640 medium supplemented with 10% HI-FBS. A total of 50,000 cells in 50 mL assay medium were added to wells of flat-bottom white 96-well plates. Fifty microliters of a mixture containing 32% fresh human serum and 2×-concentrated mAbs were added to the cells next, resulting in a final human serum concentration of 16%, and the plates were incubated at 37° C./5% CO₂ for 2 hours. Following the mAb/serum incubation, 10 mL WST-1 cell proliferation reagent (Sigma-Aldrich) were added to each well. The plate was mixed by shaking at 600 rpm for 1 min and incubated at 37° C./5% CO₂ for an additional 4 hours. Absorbance at 440 nm was used to quantify the presence of viable cells. Percentage cell viability was measured in relation to cells treated with 10 mg/mL Trastuzumab (isotype control). Percent cytotoxicity was defined as 100—(% viability). Serum from freshly drawn human blood was used as the source of human complement. Briefly, de-identified whole blood from up to 5 healthy donors (Blood Center of Brazos Valley) was collected in serum clot activator (red top) tubes, incubated upright at room temperature for 0.5-2 hours to allow clotting, and centrifuged at 1000 g for 10 minutes. The serum layers from multiple tubes were pooled, stored at 4° C., and used for the CDC assay within 24 hours of blood draw.

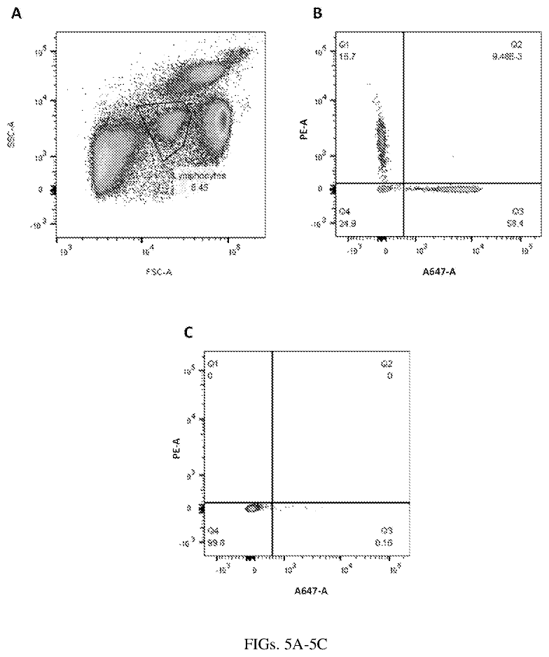

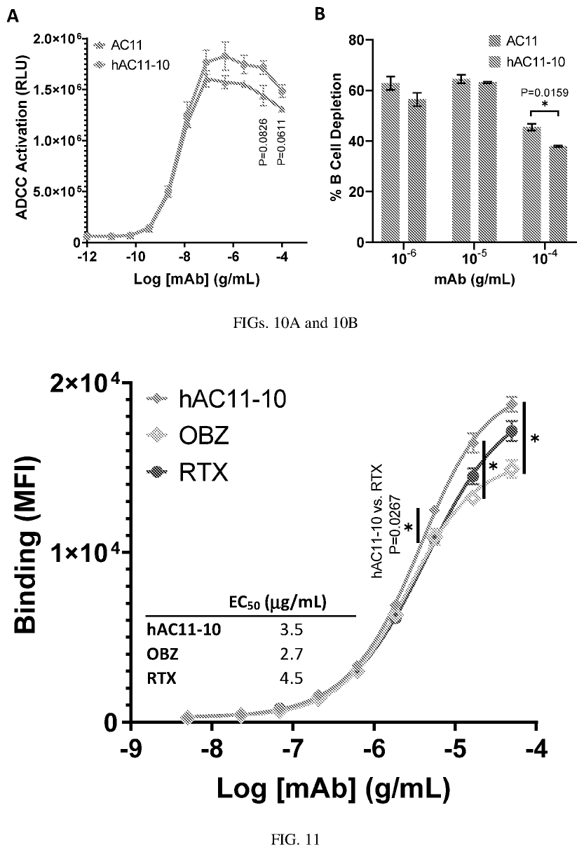

Whole-blood B cell depletion assay: Five—10 mL heparinized de-identified whole blood collected from healthy human donors (Blood Center of Brazos Valley) and stored at ambient temperature was used for the assay within 6 hours of blood draw. One hundred mL of whole blood in conical-bottom 96-well plates was mixed with 20 mL 6×-concentrated anti-hCD20 mAbs or Trastuzumab (isotype control) in Dulbecco's Phosphate Buffered Saline (DPBS)/1% BSA and the plate incubated in a humidified 37° C./5% CO₂ incubator for 24 hours. Thereafter, plates were equilibrated to ambient temperature for 10 minutes and 25 mL of the blood/mAb mixtures were combined with 25 mL of a 2×-concentrated mixture of PE-labeled anti-CD19 (B cell) and Alexa Fluor (AF647)-labeled anti-CD3 (T cell) Abs. The final concentration of the labeled Abs was 0.075 mg/mL (PE-anti-CD19) and 0.25 mg/mL (AF647-anti-CD3). The plate was incubated with rocking at ambient temperature for 30 minutes. Thereafter, the labeled Ab-blood mixtures were added to 950 mL of 1×BD FACS Lysing Solution (BD Biosciences) and incubated at ambient temperature for 10 minutes to allow lysis of red blood cells. The mixtures were centrifuged at 500 g for 5 minutes and 500 mL of the supernatant was discarded. Flow cytometric analysis was performed using a BD LSRFortessa X-20 flow cytometer. Data analysis was performed using FlowJo v10. Lymphocytes were gated based on side- and forward-light scatter characteristics (FIGS. 5A, 5B, and 5C) and 10,000 lymphocyte events were collected per sample using a high flowrate. The ratio of B cells (PE-positive) to T cells (A647-positive) was used as a normalized gauge of B cell presence. B cell depletion was calculated using the formula:

$$\% B cell depletion = 100 ( 1 - \frac{{B}_{S} {T}_{I}}{{T}_{S} {B}_{I}} )$$

where Bs/1=B cell count in mAb-treated (S) or isotype-treated (I) sample; Ts/1=T cell count in mAb-treated (S) or isotype-treated (I) sample.

Whole blood for the complement-depleted whole-blood depletion assay was prepared as follows. Heparinized whole blood obtained as described above was centrifuged at 300 g for 5 minutes at ambient temperature and the plasma layer was carefully recovered without disruption of the buffy coat containing leukocytes. For the heat-treated (HT) plasma blood, the plasma was heated at 56° C. for 30 minutes, cooled to ambient temperature for 15 minutes, and then recombined with the blood cell pellet. For the non-heat-treated (NHT) plasma blood, the plasma was kept at ambient temperature for 45 minutes prior to recombining with the blood cell pellet. Antibody-mediated B cell depletion was determined as described above.

Cross-reactivity of anti-hCD20 mAbs with mouse CD20: HEK293T cells in P100 tissue culture dishes were transfected at 70-90% confluency with 5 mg pCMV5 mCD20 expression plasmid or parental pCMV5 (mock transfection) using jetOPTIMUS (Polyplus-transfection) reagent per the manufacturer's recommended protocol at a 1:1 jetOPTIMUS:DNA (L: g) ratio. Two days post transfection, the supernatant on the transfected cells was aspirated and the cells dislodged by vigorous pipetting with DPBS (HyClone). The cells were pelleted by centrifugation and the buffer replaced with 8 mL chilled blocking buffer (DPBS/10% FBS). The cells were blocked with gentle rocking at 4° C. for 30 minutes. Two hundred mL of the blocked cells were transferred to individual wells of a conical-bottom 96-well plate and the cells were pelleted by centrifugation prior to incubation with 50 mL of 50 mg/mL A488-labeled anti-hCD20 Abs or 25 mg/mL A488-labeled anti-mouse CD20 Ab (eBioscience/Thermo Fisher) at 4° C. with rocking for 1 hour. The Ab-coated cells were washed three times with cold DPBS/1% BSA and resuspended in 300 mL DPBS/1% BSA prior to flow cytometric analysis in the FITC channel using a BD LSRFortessa X-20 cytometer. Histogram plots were generated with Flowing Software v2.5.1 (University of Turku).

SCID mouse peritoneal tumor model study: The in vivo experiments were carried out using protocols approved by the Texas A&M University Institutional Animal Care and Use Committee (IACUC #2021-0123). Raji cells (5×10⁶ cells/mL) in DPBS were labeled with 5 mM CellTrace Violet (Thermo Fisher) at ambient temperature for 7 minutes. The cells were allowed to recover in RPMI 1640 supplemented with 10% HI-FBS at 37° C./5% CO₂ for 5-7 hours. 5×10⁵ Raji cells in 100 mL DPBS were injected into the peritoneal cavity of 7-9-wk old CB-17 SCID mice (Charles River Laboratories). Sixteen hours later, Abs or DPBS (100 mL) were injected i.p. into the mice. Twenty-four hours after Ab injection, mice were sacrificed, and peritoneal lavage was performed using DPBS supplemented with 5 mM EDTA to collect the cells in the peritoneal cavity. The peritoneal cavity cells were treated with BD FACS Lysis Solution (BD Biosciences) to (i) lyse any red blood cells inadvertently collected during the lavage procedure and (ii) fix the leukocytes. Twenty mL of CountBright Plus Absolute Counting Beads (Thermo Fisher) were added to each sample prior to flow cytometric analysis to allow quantification of processed sample volumes. The number of CellTrace Violet-labeled Raji cells in each sample was quantified using a BD LSRFortessa X-20 flow cytometer. An internal DPBS treatment group was always included in experiments performed on different days. The number of remaining Raji cells in the control treatment group tended to be much lower than the number initially inoculated into the peritoneum, a phenomenon that has been previously observed, and was dependent on the specific batch of Raji cells used in a particular experiment. The low absolute number of recovered Raji cells was not due to any obvious tissue leakage, as we did not find Raji cells in the blood, spleen or lymph node. To enable data comparison between independent experiments, Raji cell counts from individual experiments were expressed as a percentage of the DPBS treatment group counts from the same experiment.

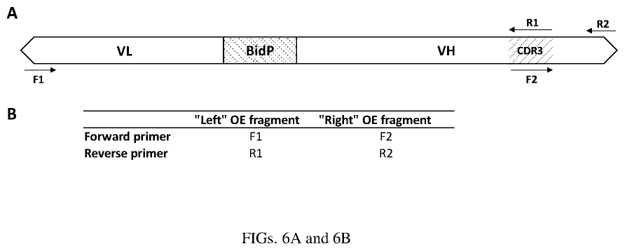

Anti-hCD20 Antibodies: The anti-hCD20 mAbs AC1 and AC11 were identified by panning a Fab-display phage library on hCD20-transfected whole cells. Clones ACR7 and ACR8 were sparsely present in the same Fab-display phage library and were subsequently identified and reconstructed through next-generation sequencing analysis (FIGS. 6A and 6B). The protein sequences of the variable regions of the chicken Abs are provided in FIG. 1 and that of the humanized Ab hAC11-10 is in FIG. 2. Chicken-derived variable heavy domains fused to the CH1 (human constant heavy) domain were inserted downstream of a mouse Ig HC V signal peptide (MGWSCIILFLVATATGVHS) and upstream of the CH2-CH3 domains from human IgG1 under the control of a CMV promoter in an in-house pCOriP expression vector derived from pCMV5. Chicken-derived variable light domains fused to a Clik (human constant light, kappa isotype) were cloned into pCOriP downstream of a mouse Ig kappa V signal peptide (METDTLLLWVLLLWVPGSTGD). pCOriP vectors for expression of the type I Abs Rituximab and Ofatumumab were similarly constructed using VH and VL gene fragments (PDB codes 2OSL and 3GIZ) synthesized by Gene Universal. Expression vectors containing the heavy and light chains of the HER2-binding mAb Trastuzumab (isotype control) were a kind gift of Dr. Sally Ward. The relevant heavy-chain and light-chain vectors were used to co-transfect Expi293F cells together with an Epstein Barr Virus Nuclear Antigen-1 expression vector, and expression/purification was performed using one-step Protein A chromatography. Protein purity was determined by SDS-PAGE and concentration measured using the Pierce BCA Protein Assay Kit (Thermo Fisher).

The type II Ab Obinutuzumab, produced in Chinese hamster ovary (CHO) cells deficient for fucosylation (a glycotype designed for maximizing ADCC), was purchased from InvivoGen (#hcd20ga-mab13).

Construction of mouse CD20 expression vector: A plasmid containing the open reading frame of mouse CD20 (mCD20; MS4A1) was purchased (OriGene Technologies MR203990). The mCD20 gene was amplified from the ORF-containing plasmid using primers incorporating the ClaI and BamHI restriction sites and the PCR product was sub-cloned into the same unique restriction sites downstream of the CMV promoter in the mammalian expression vector pCMV5. The resulting plasmid was named pCMV5 mCD20.

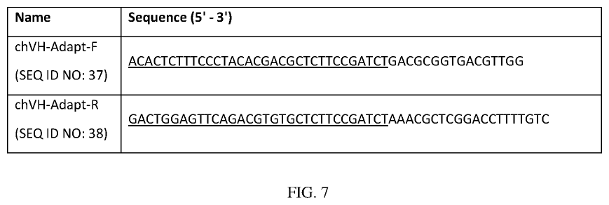

Identification and reconstruction of anti-hCD20 mAbs ACR7 and ACR8 via NGS analysis: The fourth-round-enriched phagemid pool from our Fab display phage selection was depleted of the dominant AC1 Fab clone with two restriction enzymes—NcoI and PpuMI—that uniquely cut once within CDR1 and CDR3, respectively, of the VH domain. The VH sequences from the AC1-depleted fourth-round-enriched phagemid pool were amplified using the primers chVH-Adapt-F and chVH-Adapt-R (FIG. 7) containing Illumina adapters, and deep sequencing was performed using the Amplicon-EZ service (GENEWIZ). The sequencing analysis revealed that many of the enriched VH sequences either were identical to or resembled (in one or more CDRs or some portion thereof) the dominant enriched clones AC1 or AC11. Two VH CDR3 sequences were represented at least 10 times and were not accompanied by VH CDR1/CDR2 sequences resembling the dominant clones AC1 and AC11. These two rare VH clones were named ACR7 and ACR8. The full-length Fabs ACR7 and ACR8 Fabs were constructed via overlap extension (OE) PCR with primers nested within the HCDR3 sequence in addition to flanking primers on either end of the Fab (FIGS. 6A and 6B). OE PCR fragments amplification comprised an initial 10 cycles of “linear amplification” using only a single primer—nested within HCDR3—followed by an additional 25 cycles with the inclusion of the relevant flanking primer. Phage displaying the reconstructed Fabs ACR7 and ACR8 were flow cytometrically confirmed to bind hCD20-transfected CHO cells (FIG. 4).

### Example 2

### Binding of Anti-hCD20 mAbs to B Lymphoma Cells

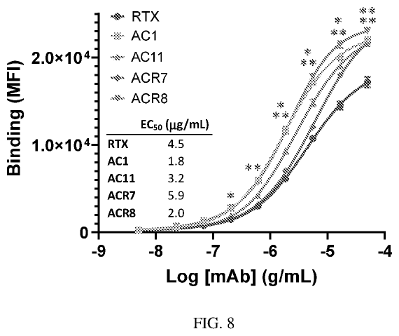

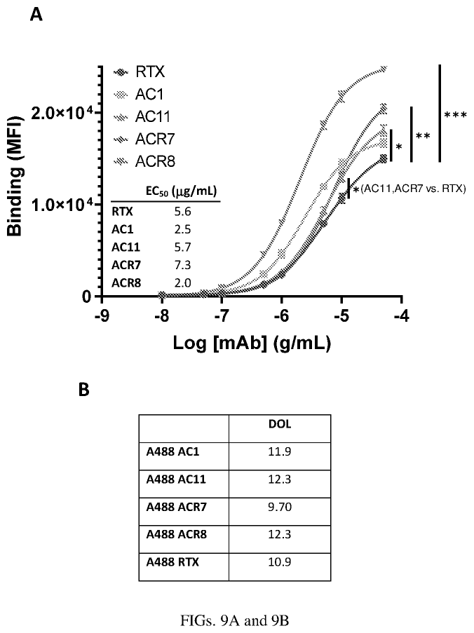

In the instant example, the binding of the chicken-derived anti-hCD20 mAbs and the control mAb Rituximab to Raji cells was evaluated using indirect immunofluorescence and flow cytometry (FIG. 8). Median fluorescence intensities from flow cytometry histograms indicating the quantity of Ab bound to cells were plotted as a function of mAb concentration. Representative flow cytometry histograms are presented in FIG. 3. The observed differences in Ab binding may derive from not only hCD20 affinity but also from the ability to occupy hCD20 sites on Raji cells dictated by epitope access. The Abs ranged in binding affinity from 12-30 nM (1.8 mg/mL-4.5 mg/mL), with all four chicken-derived mAbs showing stronger binding/higher occupancy of hCD20 on Raji cells relative to Rituximab (P<0.05) at concentrations >1.9 mg/mL. It should be noted that this data may underestimate the binding of anti-hCD20 Abs to Raji cells, particularly for strong binders at the highest mAb concentrations, due to the detection limit of the secondary anti-human IgG Fc Ab. a reproducible ˜15% weaker binding signal for 50 mg/mL ACR8 when the secondary A488 anti-human IgG Fc Ab was used at a concentration of 10 mg/mL (MFI 2.0×10⁴) vs. at 50 mg/mL (MFI 2.3×10⁴) was observed. Without being bound by any theory, these data may indicate possible sub-saturation/incomplete binding of the hCD20-bound Abs by the labeled anti-human IgG Fc Ab. Binding of anti-hCD20 Abs was also evaluated via direct immunofluorescence using Atto 488-labeled anti-hCD20 Abs, and this data showed similar overall relative binding profiles to FIG. 8, but with notably stronger binding of ACR8, particularly at high concentrations (FIGS. 9A and 9B). Data comparing the Raji cell binding/hCD20 receptor occupancy (RO) of the second-generation humanized type II anti-hCD20 mAb Obinutuzumab to a humanized version of mAb AC11, hAC11-10 (FIGS. 10A, 10B, and 2), is provided in FIG. 11.

### Example 3

### Antibody-Dependent Cellular Cytotoxicity

In the instant example, ADCC of the chicken-derived anti-hCD20 Abs relative to Rituximab was evaluated using two complementary assays. In the first method, Ab-mediated lysis of Raji cells by human PBMC effector cells carrying a mixed V158/F158 FcgRIIIa genotype was measured (FIG. 12A). Lysis was quantified by luminometric measurement of the HiBiT protein released by Raji cells. Obinutuzumab (afucosylated), which depletes B cells primarily through ADCC and direct cell-signaling effects, was included as an additional benchmark. The second experiment used an ADCC reporter bioassay and measured activation of ADCC signaling via an NFAT-responsive luciferase gene in pseudoeffector (Jurkat) cells stably expressing FcgRIIIa V158 (FIG. 12B). The four chicken-sourced anti-hCD20 Abs exhibit approximately 10-fold enhanced target cell lysis when using PBMCs as effector cells and >100-fold stronger ADCC signaling in the pseudoeffector cell bioassay, relative to Rituximab. Obinutuzumab showed the strongest Ab-mediated target cell lysis. Since, the presence of fucose at position N297, a feature normally conferred by mammalian production hosts, is known to weaken both binding to FcgRIIIa and ADCC, at least some of the ADCC advantage of the Obinutuzumab likely derives from its unique glycosylation (no fucose) profile. The discrepancy in the relative strengths of the ADCC signals measured with the PBMC effectors vs. the pseudoeffector cells may stem from the polyclonal nature of the PBMC population, which harbors a mixed V158/F158 FcgRIIIa genotype, vs. the monoclonal pseudoeffector cells (V158 FcgRIIIa only). The relatively large ADCC superiority of the anti-hCD20 mAbs to Rituximab stands in contrast to the smaller differences in Raji cell binding affinity (FIGS. 8, 9A, and 9B), suggesting that the mAbs likely bind to distinct epitopes on CD20 relative to Rituximab, giving rise to differential interplay with ADCC pathways.

Chickens represent a rich source of Ab diversity for difficult human targets, offering the possibility of complementary Abs against novel epitopes, including functional “hot spots” conserved across mammalian species. Importantly, chickens do not have a CD20 homolog, thus eliminating species-induced constraints on hCD20 epitope accessibility. In the present disclosure, four novel anti-hCD20 Abs discovered from the immune repertoire of chickens were characterized. It was discovered that all four chicken-derived Abs are superior to Rituximab in both Fc-mediated mechanisms of action—ADCC (≥10-fold) and CDC (4-8-fold). Other anti-CD20 mAbs in the clinic have tended to show superiority over Rituximab in only one of these two mechanisms. For example, the type I anti-hCD20 mAb Ofatumumab shows stronger CDC but comparable ADCC to Rituximab. On the other hand, the type 11 mAb Obinutuzumab, which is produced in a proprietary engineered cell line (Roche/GlycArt) and has a unique N-glycosylation profile comprising a bisecting N-acetylglucosamine (GlcNAc) and reduced fucosylation relative to mAbs produced in conventional production hosts, a property that confers enhanced FcgRIIIa binding and ADCC, exhibits stronger ADCC but ˜100-fold weakened CDC. Without being bound by any theory, the binding affinity of the chicken-derived mAbs (FIG. 8) may not necessarily correlate with the effector functions or whole blood B cell depletion ability, perhaps reflecting differences in the epitope and/or spatial orientation of Ab binding which can significantly modulate effector functions.

### Example 4

### Complement-Dependent Cytotoxicity

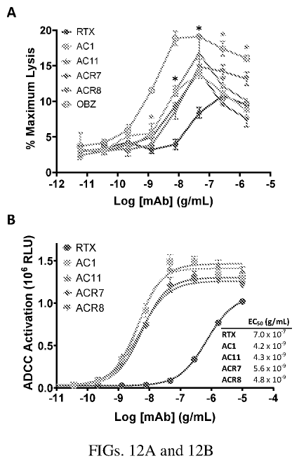

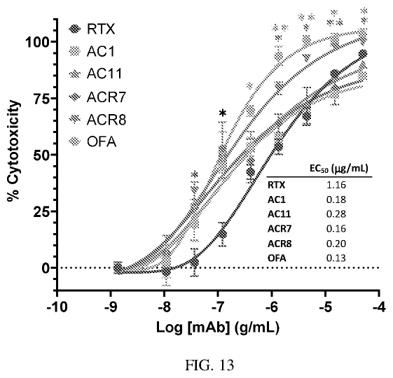

In the instant example, the ability of the four chicken-derived anti-hCD20 Abs to mediate complement-dependent cytotoxicity (CDC) in Raji cells was measured and compared to both Rituximab and a second-generation clinically used anti-hCD20 mAb known for its strong CDC, Ofatumumab (also called 2F2). Serum freshly harvested from the whole blood of healthy donors was used as the source of complement. Cytotoxicity was colorimetrically measured after incubation of cells with human serum and Abs at 37° C./5% CO₂ for 2 hours. The chicken anti-hCD20 mAbs exhibited 4-8-fold enhanced CDC relative to Rituximab and comparable CDC to Ofatumumab (FIG. 13).

### Example 5

### Whole-Blood B Cell Depletion Assay

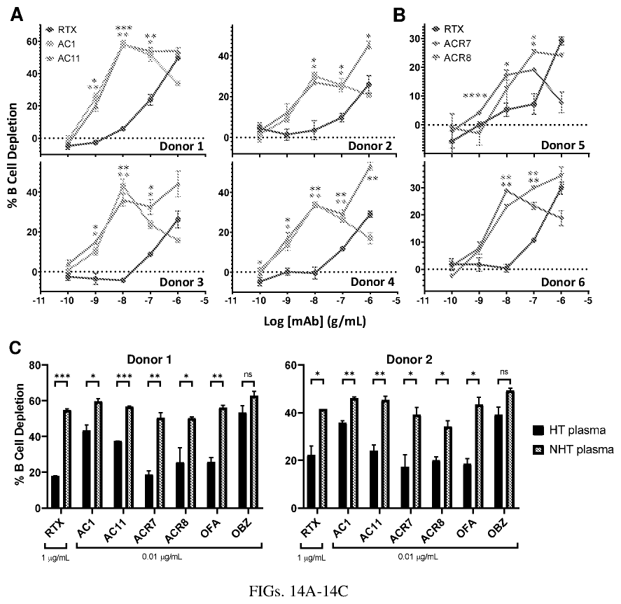

In the instant example, the ability of anti-hCD20 Abs to deplete B cells in whole blood freshly obtained from healthy human donors was evaluated. This assay provides a single in vitro readout of the outcome of multiple mechanisms of depletion within the complex whole-blood environment, including both Fc-dependent (i.e. ADCC/ADCP, CDC) and Fc-independent mechanisms such as direct cell-death signaling. Whole blood from independent human donors was used to evaluate B cell depletion of mAbs. Freshly obtained human blood was incubated with different concentrations of Abs at 37° C./5% CO₂ for 24 hours and the amount of remaining CD19-positive B cells was quantified by flow cytometry (FIGS. 14A and 14B). Relative to Rituximab, AC1 and AC11 exhibited approximately 100-fold more potent whole-blood B cell depletion, while ACR7 and ACR8 showed ˜20-100-fold enhanced B cell depletion.

To gauge the importance of complement to the B cell depletion ability of Abs in whole blood, we next performed a whole-blood B cell depletion assay in which the complement-containing plasma component of the whole blood was separated by centrifugation and was either kept at ambient temperature or heated (56° C.) to inactivate the complement prior to recombination with the original whole blood cell pellet. Heat treatment of plasma significantly dampened the B cell depletion activity of all four chicken-derived mAbs (FIG. 14C), which may indicate that complement contributes to the B-cell-specific cytotoxicity in whole blood. ACR7 and ACR8 reproducibly showed the strongest utilization of CDC, on par with both clinically used type I Abs Ofatumumab and Rituximab. In contrast, Obinutuzumab, a type II Ab characterized by strong ADCC and direct-cell-death signaling but weak complement activity, did not show a strong dependence on complement for B cell depletion in whole blood. Interestingly, Rituximab showed a significant dependence on complement for B cell depletion in a whole-blood context that was comparable to Ofatumumab, an Ab known to possess superior complement activity.

The B cell depletory action of anti-hCD20 Abs is thought to be mediated by a combination of multiple mechanisms (e.g., ADCC/ADCP, CDC, and direct cell death effects (DCDE)). While the relative contribution of these mechanisms to the overall B cell depletion outcome in vivo remains a topic of debate and varies depending on the specific Ab, ADCC/ADCP effects mediated by Fcg receptors on immune effector cells likely play an important role. The four chicken-derived mAbs exhibited a much (˜10-fold) stronger depletion of B cells in a whole blood context (FIGS. 14A and 14B) than lysis of Raji B lymphoma cells using only PBMCs as effector cells (FIG. 12A), suggesting that in the case of these Abs, immune effector cells alone are likely inadequate to account for B cell depletion activity in blood. The fact that the Abs show significantly weakened B cell depletion activity in whole blood containing heat-inactivated complement lends further support to this argument.

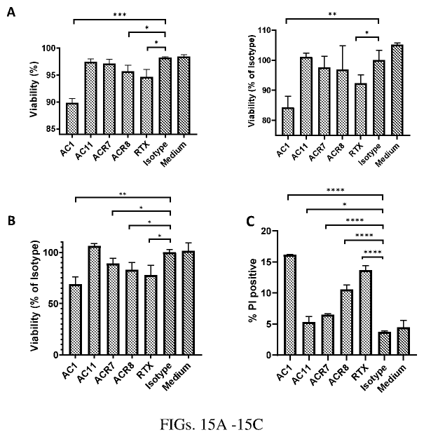

All four chicken-derived mAbs exhibit (i) strong CDC that is 4-8-fold superior to the type I Ab Rituximab (FIG. 13), (ii) complement utilization in whole blood comparable to both Rituximab and the second-generation type I anti-hCD20 Ab Ofatumumab (FIG. 14B), and (iii) little or no direct cell death effects (FIGS. 15A, 15B, and 15C), indicative of type I behavior. AC1 came closest to showing type II characteristics—specifically a comparatively weak utilization of complement in whole blood (FIG. 14B) and some direct cell death effects (FIGS. 15A, 15B, and 15C)—but its strong standalone CDC could preclude it from “pure” type II status.

### Example 6

### Cross-Reactivity of Anti-hCD20 mAbs with Mouse CD20

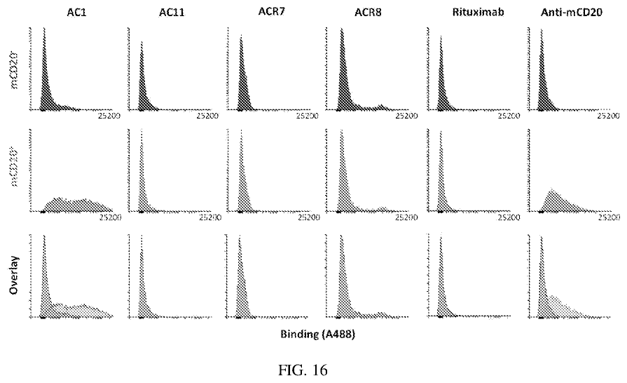

In the instant example, the ability of chicken-derived anti-hCD20 mAbs to bind mouse CD20 (mCD20) was flow cytometrically evaluated using HEK293T cells transfected with mCD20-encoding plasmid (mCD20⁺), while background binding of Abs to cells was assessed on mock-transfected (mCD20⁻) HEK293Tcells (FIG. 16). A commercially obtained anti-mCD20 Ab (eBioscience/Thermo Fisher) was used as the positive control. AC1 showed reactivity with mCD20, indicating binding to an epitope conserved between mice and humans and therefore necessarily inaccessible to current (mouse-derived) anti-hCD20 mAbs. AC1 did not show an identical mCD20 binding profile to the commercial anti-mCD20 Ab, likely due to differences in mCD20 binding affinity and/or epitope specificity. The other three chicken-derived anti-hCD20 Abs did not bind mCD20. Rituximab, an anti-hCD20 Ab isolated from mice immunized with human CD20, was unreactive with mCD20.

One of the four chicken-derived anti-hCD20 Abs, AC1, was found to bind a unique hCD20 epitope conserved between humans and mice, an epitope necessarily inaccessible to the mouse host. Given that these Abs were sourced from chickens which lack a CD20 homolog, it might seem surprising at first glance that more of the Abs did not target the 67% of CD20 sequence homologous between mice and humans. Nonetheless, in the context of the limited availability of continuous stretches of homologous sequence in the CD20 extracellular loops between humans and mice (FIG. 17), a 1-in-4 Ab hit rate for mouse CD20 cross-reactivity does not seem unusual. It is worth noting that although three of the Abs from this study are not cross-reactive with mouse CD20, it cannot be ruled out that these Abs still bind novel epitopes relative to existing mouse-derived Abs. Some portions of the CD20 sequence, particularly in the large extracellular loop, are not identical between humans and mice and yet have not represented known epitopes for mouse-derived Abs (FIG. 17), perhaps due to the high degree of cross-species homology. Chicken immunization system could provide access to antigenic regions such as these which are presumably difficult to access in the mouse host. A competitive binding experiment in which hCD20-positive Raji cells pre-incubated with unlabeled anti-hCD20 Abs were subsequently exposed to fluorescently labeled anti-hCD20 Abs. However, it was found that in this assay even Rituximab and Ofatumumab, mouse-derived anti-hCD20 mAbs which are known to bind distinct epitopes on CD20, could not bind Raji cells simultaneously, likely indicating steric/allosteric interference stemming from the compact nature of the hCD20 antigen.

### Example 7

### Humanization of mAb AC11

In the instant example, one of the chicken-human chimeric Abs was sought to be humanized, with a view to minimizing immunogenicity and extending long-term efficacy in a clinical setting. AC11 was chosen for humanization because it showed relatively high whole-blood B cell depletion activity, even at concentrations above 10⁸ g/mL (FIG. 14A). The frameworks of the human germlines IGHV3-23 and IGLV3-19 were used as templates for AC11 VH and VL complementarity-determining region (CDR) grafting. Initially, human framework residues were substituted with their parental chicken counterparts only when the calculated decrease in the T20 humanness score was negligible. This approach, however, yielded a humanized version of AC11 with no detectable hCD20-mediated in vitro cytotoxic activities. Next, we modified selected framework residues—Vernier residues that likely play a role in supporting CDR orientation and packing residues that may be non-exposed and reside at the VL/VH interface. Human residues at these positions were substituted with amino acids selected from a set comprising the parental chicken residue or residues commonly found in other chicken Abs and the human VH3 and VL3 families. The humanized AC11 variants were individually evaluated for hCD20-mediated ADCC activation ability, yielding a molecule, hAC11-10 (FIG. 2), that fully retained the ADCC activation of the parent (FIG. 10A). hAC11-10 also exhibited B cell depletion comparable to parental AC11 (FIG. 10B) and higher Raji cell binding/hCD20 occupancy relative to the clinically used 2^{nd}-generation humanized anti-hCD20 mAb Obinutuzumab at concentrations of 16.7 mg/mL and 50 mg/mL (FIG. 11). The T20 humanness scores of the humanized mAb hAC11-10 are 75.2 (VH) and 74.0 (VLI). These scores are higher than those for Rituximab of 68.3 (VH) and 65.2 (VLk), suggesting that hAC11-10 should exhibit lower immunogenicity relative to Rituximab in a human clinical setting.

### Example 8

### In Vivo Characterization in a SCID Mouse Peritoneal Tumor Model

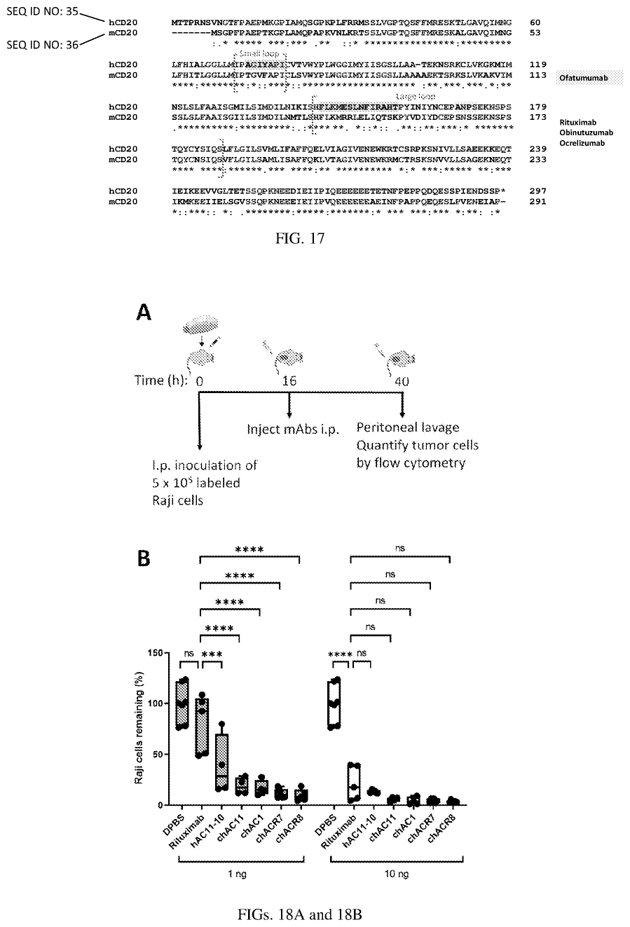

In the instant example, the ability of both the parental chicken-human chimeric Abs and humanized Ab hAC11 to deplete human Raji B lymphoma cells in vivo was evaluated in an immunocompromised mouse peritoneal tumor model at two Ab doses—1 ng and 10 ng. A similar approach was previously used to evaluate anti-CD20 Ab in vivo efficacy in C57BL/6 mice inoculated with mouse EL4 lymphoma cells stably expressing human CD20. At the 1 ng Ab dose, all the chicken-derived Abs showed significantly superior depletion of Raji cells in the mouse peritoneal cavity relative to Rituximab (FIG. 18).

All four chicken anti-hCD20 Abs and the humanized Ab hAC11-10 exhibited stronger depletion of Raji B lymphoma cells within the peritoneal cavity of SCID mice. While B lymphoma is often modeled in vivo via subcutaneously implanted solid xenografts, we reasoned that the peritoneal cavity which is rich in immune effector components may offer a complementary view, since the liquid environment does not pose the additional constraints of requiring solid tumor penetration. The SCID system has previously been used to reliably model the therapeutic action of anti-hCD20 Abs.

Loss/shaving of CD20 from tumorogenic cells has been proposed as one mechanism of relapse in CD20-mediated immunotherapies and suggests that anti-hCD20 Abs with stronger effector functions alone may not guarantee improved clinical outcome.

Nonetheless, anti-hCD20 mAbs with complementary modes of action and/or stronger effector functions relative to Rituximab in vitro have previously proven effective against Rituximab resistance/relapse both in vitro and in clinical settings.

1. An antigen-binding molecule that specifically binds to CD20, wherein the antigen-binding molecule comprises an immunoglobulin heavy chain variable domain (VH) and an immunoglobulin light chain variable domain (VL), wherein the VH comprises a complementarity determining region 1 (VH CDR1) comprising the amino acid sequence of SEQ ID NO: 9, a VH CDR2 comprising the amino acid sequence of SEQ ID NO: 13 and a VH CDR3 comprising the amino acid sequence of SEQ ID NO: 17; and wherein the VL comprises a complementarity determining region 1 (VL CDR1) comprising the amino acid sequence of SEQ ID NO: 21, a VL CDR2 comprising the amino acid sequence of SEQ ID NO: 25, and a VL CDR3 comprising the amino acid sequence of SEQ ID NO: 29:
VH CDR1
(SEQ ID NO: 9)
SSYAM
VH CDR2
(SEQ ID NO: 13)
EITNYAAGSGTWYGAAVK
VH CDR3
(SEQ ID NO: 17)
KGPISGSIGYLSSIDA
VL CDR1
(SEQ ID NO: 21)
SGGSSRYGYG
VL CDR2
(SEQ ID NO: 25)
WNDKRPS
VL CDR3
(SEQ ID NO: 29)
GNYDNNDPA.

2. The antigen-binding molecule of claim 1, wherein the VH comprises an amino acid sequence having at least 80% sequence identity to SEQ ID NO: 1.

3.-13. (canceled)

14. The antigen-binding molecule of claim 1, wherein the VL comprises an amino acid sequence having at least 80% sequence identity to SEQ ID NO: 5.

15.-25. (canceled)

26. An antigen-binding molecule that specifically binds to CD20, wherein the antigen-binding molecule comprises an immunoglobulin heavy chain variable domain (VH) and an immunoglobulin light chain variable domain (VL), wherein the VH comprises a complementarity determining region 1 (VH CDR1) comprising the amino acid sequence of SEQ ID NO: 10, a VH CDR2 comprising the amino acid sequence of SEQ ID NO: 14 and a VH CDR3 comprising the amino acid sequence of SEQ ID NO: 18; and wherein the VL comprises a complementarity determining region 1 (VL CDR1) comprising the amino acid sequence of SEQ ID NO: 22, a VL CDR2 comprising the amino acid sequence of SEQ ID NO: 26, and a VL CDR3 comprising the amino acid sequence of SEQ ID NO: 30:
VH CDR1
(SEQ ID NO: 10)
SSSYIN
VH CDR2
(SEQ ID NO: 14)
QINKDGGGSWYAPVVK
VH CDR3
(SEQ ID NO: 18)
KNADSGCVGVGGCIDT
VL CDR1
(SEQ ID NO: 22)
SGGSYYYGGNYYYG
VL CDR2
(SEQ ID NO: 26)
NNNKRPS
VL CDR3
(SEQ ID NO: 30)
GGYDSSYVAI.

27. The antigen-binding molecule of claim 26, wherein the VH comprises an amino acid sequence having at least 80% sequence identity to SEQ ID NO: 2.

28.-38. (canceled)

39. The antigen-binding molecule of claim 26, wherein the VL comprises an amino acid sequence having at least 80% sequence identity to SEQ ID NO: 6.

40.-50. (canceled)

51. The antigen-binding molecule of claim 26, wherein the VH comprises an amino acid sequence having at least 80% sequence identity to SEQ ID NO: 33.

52.-62. (canceled)

63. The antigen-binding molecule of claim 26, wherein the VL comprises an amino acid sequence having at least 80% sequence identity to SEQ ID NO: 34.

64.-74. (canceled)

75. An antigen-binding molecule that specifically binds to CD20, wherein the antigen-binding molecule comprises an immunoglobulin heavy chain variable domain (VH) and an immunoglobulin light chain variable domain (VL), wherein the VH comprises a complementarity determining region 1 (VH CDR1) comprising the amino acid sequence of SEQ ID NO: 11, a VH CDR2 comprising the amino acid sequence of SEQ ID NO: 15 and a VH CDR3 comprising the amino acid sequence of SEQ ID NO: 19; and wherein the VL comprises a complementarity determining region 1 (VL CDR1) comprising the amino acid sequence of SEQ ID NO: 23, a VL CDR2 comprising the amino acid sequence of SEQ ID NO: 27, and a VL CDR3 comprising the amino acid sequence of SEQ ID NO: 31:
VH CDR1
(SEQ ID NO: 11)
NSNGMG
VH CDR2
(SEQ ID NO: 15)
GIHSSGRYTYYGTAVK
VH CDR3
(SEQ ID NO: 19)
KNADSAYGYWYAGSIDA
VL CDR1
(SEQ ID NO: 23)
SGGYSSYGYS
VL CDR2
(SEQ ID NO: 27)
NNNNRPS
VL CDR3
(SEQ ID NO: 31)
AFTDYSSLAGV.

76. The antigen-binding molecule of claim 75, wherein the VH comprises an amino acid sequence having at least 80% sequence identity to SEQ ID NO: 3.

77.-87. (canceled)

88. The antigen-binding molecule of claim 75, wherein the VL comprises an amino acid sequence having at least 80% sequence identity to SEQ ID NO: 7.

89.-124. (canceled)

125. The antigen-binding molecule of claim 1, wherein the antigen-binding molecule is an antibody or a CD20-binding fragment thereof.

126. The antigen-binding molecule of claim 125, wherein the CD20-binding fragment is selected from the group consisting of an Fab fragment, an scFab, an Fab′, a single chain variable fragment (scFv) and a one-armed antibody.

127. The antigen-binding molecule of claim 1, wherein the antigen-binding molecule is a humanized antibody or CD20-binding fragment thereof.

128.-146. (canceled)

147. The antigen-binding molecule of claim 26, wherein the antigen-binding molecule is an antibody or a CD20-binding fragment thereof.

148. The antigen-binding molecule of claim 147, wherein the CD20-binding fragment is selected from the group consisting of an Fab fragment, an scFab, an Fab′, a single chain variable fragment (scFv) and a one-armed antibody.

149. The antigen-binding molecule of claim 26, wherein the antigen-binding molecule is a humanized antibody or CD20-binding fragment thereof.

150. The antigen-binding molecule of claim 75, wherein the antigen-binding molecule is an antibody or a CD20-binding fragment thereof.

151. The antigen-binding molecule of claim 150, wherein the CD20-binding fragment is selected from the group consisting of an Fab fragment, an scFab, an Fab′, a single chain variable fragment (scFv) and a one-armed antibody.

152. The antigen-binding molecule of claim 75, wherein the antigen-binding molecule is a humanized antibody or CD20-binding fragment thereof.
